(19) United States
(12) Patent Application Publication
(10) Pub. No.: US 2023/0399647 A1
(43) Pub. Date: Dec. 14, 2023

(54) ENGINEERED BIOSYNTHETIC PATHWAYS FOR PRODUCTION OF ECTOINE BY FERMENTATION

(71) Applicant: Zymergen Inc., Emeryville, CA (US)

(72) Inventors: Stepan Tymoshenko, Emeryville, CA (US); Anupam Chowdhury, Emeryville, CA (US); Zhihao Wang, Emeryville, CA (US); Jeffrey Mellin, Emeryville, CA (US); Michael Shareef Siddiqui, Oakland, CA (US); Cara Ann Tracewell, Walnut Creek, CA (US); Alexander Glennon Shearer, San Francisco, CA (US); Hannah Edstrom, Emeryville, CA (US); Michael Blaisse, Emeryville, CA (US)

(21) Appl. No.: 17/917,549

(22) PCT Filed: Feb. 11, 2021
(86) PCT No.: PCT/US21/17696

§ 371 Date: Oct. 6, 2022

(51) Int. Cl. C12N 15/52; C12N 9/04; C12N 9/02; C12N 9/88; C12P 17/12; C12P 13/04; C12N 9/10; C12N 9/12

(52) U.S. Cl. CPC C12N 15/52; C12Y 402/01108; C12Y 207/02004; C12N 9/0006; C12Y 206/01001; C12N 9/0008; C12Y 101/01037; C12N 9/88; C12P 17/12; C12P 13/04; C12N 9/1096; C12N 9/1217; C12Y 102/01011

(57) ABSTRACT

The present disclosure describes the engineering of microbial cells for fermentative production of ectoine and provides novel engineered microbial cells and cultures, as well as related ectoine production methods.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING

This application includes a sequence listing which has been submitted electronically in ASCII format and is hereby incorporated by reference in its entirety. This ASCII copy, created on Jan. 25, 2021, is named ZMGNP034WO_SL.txt. and is 109,752 bytes in size.

### FIELD OF THE DISCLOSURE

The present disclosure relates generally to the area of engineering microbes for production of ectoine by fermentation.

### BACKGROUND

Ectoine (1,4,5,6-tetrahydro-2-methyl-4-pyrimidinecarboxylic acid) is a natural compound found in several species of bacteria. It is a compatible solute which serves as a protective substance by acting as an osmolyte and thus helps organisms survive extreme osmotic stress. Ectoine is found in high concentrations in halophilic microorganisms and confers resistance towards salt and temperature stress. Ectoine was first identified in the microorganism Ectothiorhodospira halochloris.

Ectoine is synthesized in three successive enzymatic reactions starting from L-aspartate-4-semialdehyde. The genes involved in the biosynthesis are called ectA, ectB and ectC and they encode the enzymes L-2,4-diaminobutyric acid acetyltransferase, L-2,4-diaminobutyric acid transaminase (“diaminobutyrate aminotransferase”), and ectoine synthase, respectively.

Ectoine is used as an active ingredient in skin care and sun protection products. It stabilizes proteins and other cellular structures and protects the skin from stresses like UV irradiation and dryness.

### SUMMARY

Various embodiments contemplated herein may include, but need not be limited to, one or more of the following:

Embodiment 1: An engineered microbial cell that produces diaminobutyric acid, wherein the engineered microbial cell expresses a non-native diaminobutyrate aminotransferase.

Embodiment 2: The engineered microbial cell of embodiment 1, wherein the engineered microbial cell expresses a non-native L-2,4-diaminobutyrate acetyltransferase and produces N-acetyl-2,4-diaminobutyrate.

Embodiment 3: The engineered microbial cell of embodiment 2, wherein the engineered microbial cell expresses a non-native ectoine synthase and produces ectoine.

Embodiment 4: The engineered microbial cell of any one of embodiments 1-3, that comprises increased activity of at least one or more upstream pathway enzyme(s) leading to L-aspartate-4-semialdehyde, said increased activity being increased relative to a control cell.

Embodiment 5: The engineered microbial cell of embodiment 4, wherein the one or more upstream pathway enzyme(s) are selected from the group consisting of aspartate semi-aldehyde dehydrogenase, apartokinase, aspartate aminotransferase, diaminobutyrate aminotransferase, and malate dehydrogenase.

Embodiment 6: An engineered microbial cell that produces diaminobutyric acid, wherein the engineered microbial cell comprises means for expressing a non-native diaminobutyrate aminotransferase.

Embodiment 7: The engineered microbial cell of embodiment 6, wherein the engineered microbial cell comprises means for expressing a non-native L-2,4-diaminobutyrate acetyltransferase and produces N-acetyl-2,4-diaminobutyrate.

Embodiment 8: The engineered microbial cell of embodiment 7, wherein the engineered microbial cell comprises means for expressing a non-native ectoine synthase and produces ectoine.

Embodiment 9: The engineered microbial cell of any one of embodiments 6-8, that comprises means for increasing the activity of at least one or more upstream pathway enzymes leading to L-aspartate-4-semialdehyde, said increased activity being increased relative to a control cell.

Embodiment 10: The engineered microbial cell of embodiment 9, wherein the one or more upstream pathway enzyme(s) are selected from the group consisting of aspartate semi-aldehyde dehydrogenase, apartokinase, aspartate aminotransferase, diaminobutyrate aminotransferase, and malate dehydrogenase.

Embodiment 11: The engineered microbial cell of any one of embodiments 1-10, wherein the engineered microbial cell is a bacterial cell.

Embodiment 12: The engineered microbial cell of embodiment 11, wherein the bacterial cell is a cell of the genus Corynebacterium.

Embodiment 13: The engineered microbial cell of embodiment 12, wherein the bacterial cell is a cell of the species glutamicum.

Embodiment 14: The engineered microbial cell of embodiment 13, wherein the non-native diaminobutyrate aminotransferase has at least 70% amino acid sequence identity with: a Glaesserella parasuis MN-H diaminobutyrate aminotransferase comprising SEQ ID NO:1; and/or a Pseudomonas putida diaminobutyrate aminotransferase comprising SEQ ID NO:2.

Embodiment 15: The engineered microbial cell of embodiment 14, wherein: the Glaesserella parasuis MN-H diaminobutyrate aminotransferase comprises SEQ ID NO:1; and/or the Pseudomonas putida diaminobutyrate aminotransferase comprises SEQ ID NO:2.

Embodiment 16: The engineered microbial cell of any one of embodiments 1-15, wherein, when cultured, the engineered microbial cell produces diaminobutyric acid at a level of at least 500 mg/L of culture medium.

Embodiment 17: The engineered microbial cell of any one of embodiments 14-16, wherein the engineered microbial cell expresses a non-native L-2,4-diaminobutyrate acetyltransferase and produces N-acetyl-2,4-diaminobutyrate.

Embodiment 18: The engineered microbial cell of embodiment 17 wherein the non-native a L-2,4-diaminobutyrate acetyltransferase has at least 70% amino acid sequence identity with: a Methylomicrobium alcaliphilum (strain DSM 19304/NCIMB 14124/VKM B-2133/20Z) L-2,4-diaminobutyrate acetyltransferase comprising SEQ ID NO:3; and/or a Desulfurispirillum indicum (strain ATCC BAA-1389/S5) L-2,4-diaminobutyrate acetyltransferase comprising SEQ ID NO:4.

Embodiment 19: The engineered microbial cell of embodiment 18, wherein: the Methylomicrobium alcaliphilum (strain DSM 19304/NCIMB 14124/VKM B-2133/20Z) L-2,4-diaminobutyrate acetyltransferase comprises SEQ ID NO:3; and/or the Desulfurispirillum indicum (strain ATCC BAA-1389/S5) L-2,4-diaminobutyrate acetyltransferase comprises SEQ ID NO:4.

Embodiment 20: The engineered microbial cell of embodiment 18 or embodiment 19, wherein the engineered microbial cell expresses a non-native ectoine synthase and produces ectoine.

Embodiment 21: The engineered microbial cell of embodiment 20, wherein the non-native ectione synthase has at least 70% amino acid sequence identity with: a hydrothermal vent metagenome ectione synthase comprising SEQ ID NO:5; and/or an Alkalilimnicola ehrlichii (strain ATCC BAA-1101/DSM 17681/MLHE-1) ectione synthase comprising SEQ ID NO:6.

Embodiment 22: The engineered microbial cell of embodiment 21, wherein: the hydrothermal vent metagenome ectione synthase comprises SEQ ID NO:5; and/or the Alkalilimnicola ehrlichii (strain ATCC BAA-1101/DSM 17681/MLHE-1) ectione synthase comprises SEQ ID NO:6.

Embodiment 23: The engineered microbial cell of embodiment 22, wherein: the hydrothermal vent metagenome ectione synthase comprises SEQ ID NO:5, and the non-native L-2,4-diaminobutyrate acetyltransferase comprises a Methylomicrobium alcaliphilum (strain DSM 19304/NCIMB 14124/VKM B-2133/20Z) L-2,4-diaminobutyrate acetyltransferase comprising SEQ ID NO:3; and/or the Alkalilimnicola ehrlichii (strain ATCC BAA-1101/DSM 17681/MLHE-1) ectione synthase comprises SEQ ID NO:6, and the non-native L-2,4-diaminobutyrate acetyltransferase comprises a Desulfurispirillum indicum (strain ATCC BAA-1389/S5) L-2,4-diaminobutyrate acetyltransferase comprising SEQ ID NO:4.

Embodiment 24: The engineered microbial cell of embodiment 23, wherein, when cultured, the engineered microbial cell produces ectoine at a level of at least 500 mg/L of culture medium.

Embodiment 25: A culture of engineered microbial cells according to any one of embodiments 1-24.

Embodiment 26: The culture of embodiment 25, wherein the culture comprises diaminobutyric acid.

Embodiment 27: The culture of embodiment 26, wherein the culture comprises diaminobutyric acid at a level of at least 500 mg/L of culture medium.

Embodiment 28: The culture of embodiment 26 or embodiment 27, wherein the culture comprises N-acetyl-2,4-diaminobutyrate.

Embodiment 29: The culture of embodiment 28, wherein the culture comprises N-acetyl-2,4-diaminobutyrate at a level of at least 500 mg/L of culture medium.

Embodiment 30: The culture of embodiment 28 or embodiment 29, wherein the culture comprises ectoine.

Embodiment 31: The culture of embodiment 30, wherein the culture comprises ectoine at a level of at least 500 mg/L of culture medium.

Embodiment 32: A method of culturing engineered microbial cells according to any one of embodiments 1-24, the method comprising culturing the cells under conditions suitable for producing diaminobutyric acid.

Embodiment 33: The method of embodiment 32, wherein the engineered microbial cells produce diaminobutyric acid at a level of at least 500 mg/L of culture medium.

Embodiment 34: The method of embodiment 33, wherein the engineered microbial cells produce N-acetyl-2,4-diaminobutyrate at a level of at least 500 mg/L of culture medium.

Embodiment 35: The method of embodiment 34, wherein the engineered microbial cells produce ectoine at a level of at least 500 mg/L of culture medium.

Embodiment 36: The method of any one of embodiments 33-35, wherein the method additionally comprises recovering diaminobutyric acid, N-acetyl-2,4-diaminobutyrate, or ectoine, respectively, from the culture.

### BRIEF DESCRIPTION OF THE DRAWINGS

FIG. 1: Biosynthetic pathway for ectoine.

FIG. 2: Diaminobutyric acid titers measured in the extracellular broth following fermentation by first-round engineered host Corynebacterium glutamicum, which was engineered to produce diaminobutyric acid.

FIG. 3: Diaminobutyric acid titers measured in the extracellular broth following fermentation by second-round engineered host C. glutamicum.

FIG. 4: Diaminobutyric acid titers measured in the extracellular broth following fermentation by third-round engineered host C. glutamicum.

FIG. 5: Ectoine titers measured in the extracellular broth following fermentation by C. glutamicum engineered to produce ectoine.

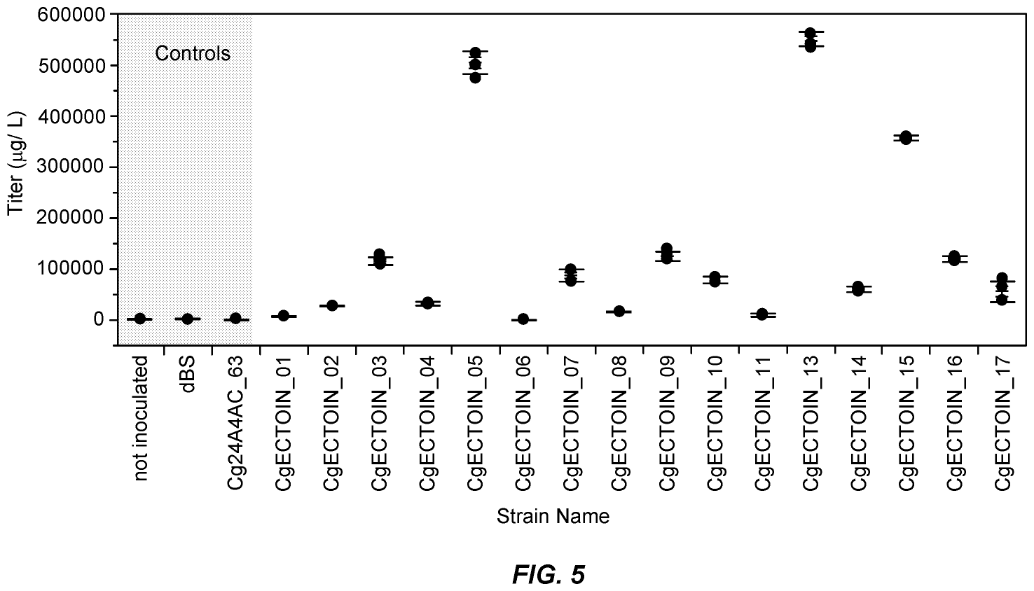

FIG. 6: Integration of Promoter-Gene-Terminator into Saccharomyces cerevisiae.

FIG. 7: Integration of Promoter-Gene-Terminator into Corynebacterium glutamicum.

### DETAILED DESCRIPTION

This disclosure describes a method for the production of the small molecule ectoine via fermentation by a microbial host from simple carbon and nitrogen sources, such as glucose and urea, respectively. This aim was achieved via the introduction of a non-native metabolic pathway into a suitable microbial host for industrial fermentation of large-scale chemical products, such as Corynebacterium glutamicum. The engineered metabolic pathway links the central metabolism of the host to the non-native pathway to enable the production of ectoine. The simplest embodiment of this method is the expression of three non-native enzymes, diaminobutyrate aminotransferase, L-2,4-diaminobutyric acid acetyltransferase, and ectoine synthase, in the microbial host.

### Definitions

Terms used in the claims and specification are defined as set forth below unless otherwise specified.

The term “fermentation” is used herein to refer to a process whereby a microbial cell converts one or more substrate(s) into a desired product (such as ectoine) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the enzyme native to the cell or a form of the enzyme that is native to the cell, but is naturally less sensitive to feedback inhibition than one or more other natural forms of the enzyme. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “ectoine” refers to a chemical compound of the formula C₆H₁₀N₂O₂ (CAS #96702-03-3; IUPAC name (S)-2-methyl-3,4,5,6-tetrahydropyrimidine-4-carboxylic acid).

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., ectoine) produced by a culture of microbial cells divided by the culture volume.

As used herein with respect to recovering ectoine from a cell culture, “recovering” refers to separating the ectoine from at least one other component of the cell culture medium.

### Engineering Microbes for Ectoine Production

Ectoine Biosynthesis Pathway

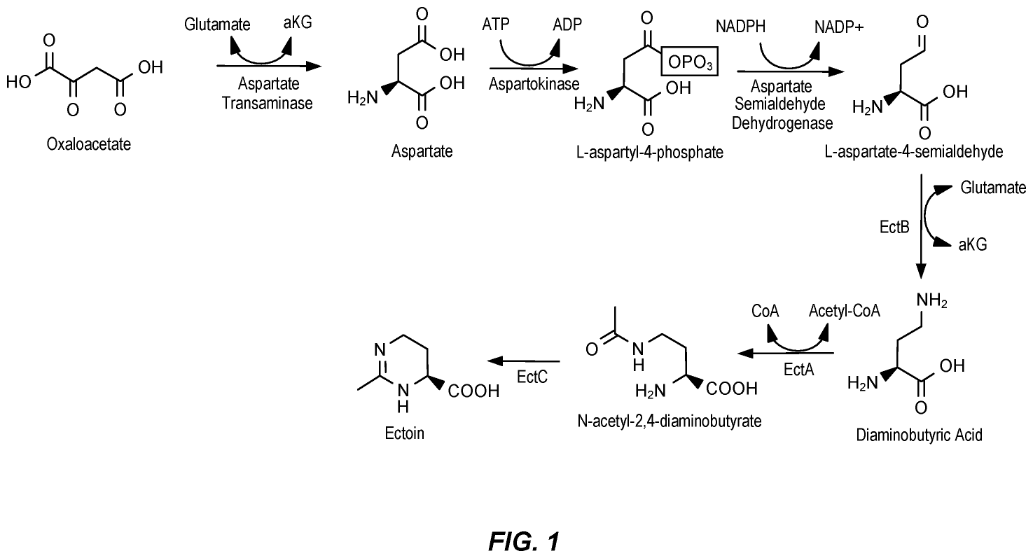

Ectoine can be produced from L-aspartate semi-aldehyde in three enzymatic steps, requiring the enzymes diaminobutyrate aminotransferase, L-2,4-diaminobutyric acid transaminase, and ectoine synthase. The ectoine biosynthesis pathway is shown in FIG. 1. Accordingly, a microbial host that can produce the precursors L-aspartate-4-semialdehyde can be engineered to produce ectoine by expressing forms of a diaminobutyrate aminotransferase, an L-2,4-diaminobutyric acid transaminase, and an ectoine synthase that are active in the microbial host. These enzymes produce diaminobutyric acid, N-acetyl-2,4-diaminobutyrate, and ectoine, respectively. For ease of discussion, the following description focuses on ectoine production. However, for hosts that do not naturally produce the ectoine precursors diaminobutyric acid and N-acetyl-2,4-diaminobutyrate, those of skill in the art understand that the description below (e.g., relating to titers, etc.) applies equally to diaminobutyric acid and N-acetyl-2,4-diaminobutyrate.

Engineering for Microbial Ectoine Production

Any diaminobutyrate aminotransferase, L-2,4-diaminobutyric acid transaminase, and ectoine synthase that is active in the microbial cell being engineered may be introduced into the cell, typically by introducing and expressing the gene(s) encoding the enzyme(s)s using standard genetic engineering techniques. Suitable diaminobutyrate aminotransferases, L-2,4-diaminobutyric acid transaminases, and ectoine synthases may be derived from any source, including plant, archaeal, fungal, gram-positive bacterial, and gram-negative bacterial sources.

One or more copies of any of these genes can be introduced into a selected microbial host cell. If more than one copy of a gene is introduced, the copies can have the same or different nucleotide sequences. In some embodiments, one or both (or all) of the non-native gene(s) is/are expressed from a strong, constitutive promoter. In some embodiments, the non-native gene(s) is/are expressed from an inducible promoter. The non-native gene(s) can optionally be codon-optimized to enhance expression in the selected microbial host cell. The codon-optimization tables used in the Examples are as follows: Bacillus subtilis Kazusa codon table: www.kazusa.orjp/codon/cgi-bin/showcodon.cgi?species=1423&aa=1&style=N; Yarrowia lipolytica Kazusa codon table: www.kazusa.orjp/codon/cgi-bin/showcodon.cgi?species=4952&aa=1&style=N; Corynebacterium glutamicum Kazusa codon table: www.kazusa.or.jp/codon/cgi-bin/showcodon.cgi?species=340322&aa=1&style=N; Saccharomyces cerevisiae Kazusa codon table: www.kazusa.or.jp/codon/cgi-bin/showcodon.cgi?species=4932&aa=1&style=N. Also used, was a modified, combined codon usage scheme for S. cerevisiae and C. glutamicum, which is reproduced below.

Modified Codon Usage Table for Sc and Cg

| Amino | | |
|---|---|---|
| Acid | Codon | Fraction |
| A | GCG | 0.22 |
| A | GCA | 0.29 |
| A | GCT | 0.24 |
| A | GCC | 0.25 |
| C | TGT | 0.36 |
| C | TGC | 0.64 |
| D | GAT | 0.56 |
| D | GAC | 0.44 |
| E | GAG | 0.44 |
| E | GAA | 0.56 |
| F | TTT | 0.37 |
| F | TTC | 0.63 |
| G | GGG | 0.08 |
| G | GGA | 0.19 |
| G | GGT | 0.3 |
| G | GGC | 0.43 |
| H | CAT | 0.32 |
| H | CAC | 0.68 |
| I | ATA | 0.03 |
| I | ATT | 0.38 |
| I | ATC | 0.59 |
| K | AAG | 0.6 |
| K | AAA | 0.4 |
| L | TTG | 0.29 |
| L | TTA | 0.05 |
| L | CTG | 0.29 |
| L | CTA | 0.06 |
| L | CTT | 0.17 |
| L | CTC | 0.14 |
| M | ATG | 1 |
| N | AAT | 0.33 |
| N | AAC | 0.67 |
| P | CCG | 0.22 |
| P | CCA | 0.35 |
| P | CCT | 0.23 |
| P | CCC | 0.2 |
| Q | CAG | 0.61 |
| Q | CAA | 0.39 |
| R | AGG | 0.11 |
| R | AGA | 0.12 |
| R | CGG | 0.09 |
| R | CGA | 0.17 |
| R | CGT | 0.34 |
| R | CGC | 0.18 |
| S | AGT | 0.08 |
| S | AGC | 0.16 |
| S | TCG | 0.12 |
| S | TCA | 0.13 |
| S | TCT | 0.17 |
| S | TCC | 0.34 |
| T | ACG | 0.14 |
| T | ACA | 0.12 |
| T | ACT | 0.2 |
| T | ACC | 0.53 |
| V | GTG | 0.36 |
| V | GTA | 0.1 |
| V | GTT | 0.26 |
| V | GTC | 0.28 |
| W | TGG | 1 |
| Y | TAT | 0.34 |
| Y | TAC | 0.66 |

### Increasing the Activity of Upstream Enzymes

One approach to increasing ectoine production in a microbial cell that is capable of such production is to increase the activity of one or more upstream enzymes leading to the precursor L-aspartate-4-semialdehyde. Upstream pathway enzymes include all enzymes involved in the conversions from a feedstock all the way to L-aspartate-4-semialdehyde. Illustrative enzymes, for this purpose, include, but are not limited to, those shown in FIG. 1 in the pathways leading to these precursors. Suitable upstream pathway genes encoding these enzymes may be derived from any available source, including, for example, those disclosed herein.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

Alternatively, or in addition, one or more promoters can be substituted for native promoters. In certain embodiments, the replacement promoter is stronger than the native promoter and/or is a constitutive promoter.

In some embodiments, the activity of one or more upstream pathway enzymes is supplemented by introducing one or more of the corresponding genes into the engineered microbial host cell. An introduced upstream pathway gene may be from an organism other than that of the host cell or may simply be an additional copy of a native gene. In some embodiments, one or more such genes are introduced into a microbial host cell capable of ectoine production and expressed from a strong constitutive promoter and/or can optionally be codon-optimized to enhance expression in the selected microbial host cell.

Titers

In various embodiments, the ectoine titers achieved by the genetic engineering methods described herein are at least 100, 200, 300, 400, 500, 600, 700, 800, or 900 mg/L or at least 1, 1.5, 2, 2.5, 3, 3.5, 4, 4.5, 5, 10, 15, 20, or 25 gm/L. In various embodiments, the titer is in the range of 100 mg/L to 25 gm/L, 300 mg/L to 10 gm/L, 500 mg/L to 5 gm/L, 600 mg/L to 4 gm/L, 700 mg/L to 3 gm/L, 800 mg/L to 2 gm/L, 900 mg/L to 1.5 gm/L, or any range bounded by any of the values listed above.

Illustrative Amino Acid and Nucleotide Sequences

The following table identifies amino acid and nucleotide sequences used in Examples. The corresponding sequences are shown in the Sequence Listing.

SEQ ID NO Cross-Reference Table

Enzyme Description (genus and species, activity name, EC identifier, UniProt identifier)

| | AA SEQ ID NO |
|---|---|
| Glaesserella parasuis, Diaminobutyrate--2-oxoglutarate aminotransferase, 2.6.1.76, U4RVR1 | 1 |
| Pseudomonas putida, Diaminobutyrate-2-oxoglutarate transaminase, 2.6.1.76, Q88F75 | 2 |
| Methylomicrobium alcaliphilum, L-2,4-diaminobutyric acid acetyltransferase, 2.3.1.178, Q4JQJ5 | 3 |
| Desulfurispirillum indicum, L-2,4-diaminobutyric acid acetyltransferase, 2.3.1.178, E6W3G0 | 4 |
| hydrothermal vent metagenome, L-ectoine synthase, 4.2.1.108, A0A160TCS0 | 5 |
| Alkalilimnicola ehrlichii, L-ectoine synthase, 4.2.1.108, Q0A9E8 | 6 |
| Methanobacterium formicicum, L-2,4-diaminobutyric acid acetyltransferase EctA, 2.3.1.178, A0A090I5X2 | 7 |
| Hahella chejuensis, L-ectoine synthase, 4.2.1.108, Q2SLV8 | 8 |
| Methanosaeta sp. SDB, L-2,4-diaminobutyric acid acetyltransferase, 2.3.1.178, A0A0Q1AWK1 | 9 |
| Phenylobacterium zucineum, L-ectoine synthase, 4.2.1.108, B4R981 | 10 |
| Methanothrix soehngenii, L-2,4-diaminobutyric acid acetyltransferase, 2.3.1.178, F4C028 | 11 |
| Bacillus halodurans, L-ectoine synthase, 4.2.1.108, Q9KED5 | 12 |
| Nitrosopumilus maritimus, L-2,4-diaminobutyric acid acetyltransferase, 2.3.1.178, A9A2K3 | 13 |
| Sphingopyxis alaskensis, L-ectoine synthase, 4.2.1.108, Q1GNW6 | 14 |
| Candidatus Entotheonella sp., Uncharacterized protein, 4.2.1.108, W4LG62 | 15 |
| Flexistipes sinusarabici, L-2,4-diaminobutyric acid acetyltransferase, 2.3.1.178, F8E4K4 | 16 |
| Leptospirillum ferriphilum YSK, L-2,4-diaminobutyric acid acetyltransferase, 2.3.1.178, A0A059XSI6 | 17 |
| Nitrincola nitratireducens, L-ectoine synthase, 4.2.1.108, W9UWT5 | 18 |
| Nitrospina sp. SCGC_AAA799_A02, L-2,4-diaminobutyric acid acetyltransferase, 2.3.1.178, A0A0J6YS11 | 19 |
| Marinobacterium stanieri, L-ectoine synthase, 4.2.1.108, A0A1N6PYR6 | 20 |
| Spirochaeta africana, L-2,4-diaminobutyric acid acetyltransferase, 2.3.1.178, H9UMW7 | 21 |
| Actinomadura sp. CNU-125, L-ectoine synthase, 4.2.1.108, A0A1Q9TDA5 | 22 |
| Dehalogenimonas lykanthroporepellens, L-2,4-diaminobutyric acid acetyltransferase, 2.3.1.178, D8K1W8 | 23 |
| Leptospirillum ferriphilum YSK, L-ectoine synthase, 4.2.1.108, A0A059XT79 | 24 |
| Bordetella bronchiseptica, L-2,4-diaminobutyric acid acetyltransferase, 2.3.1.178, Q7WHI7 | 25 |
| Betaproteobacteria bacterium SG8_41, L-ectoine synthase, 4.2.1.108, A0A0S8C710 | 26 |
| hydrothermal vent metagenome, L-ectoine synthase, 4.2.1.108, A0A160TXJ7 | 27 |
| Deltaproteobacteria bacterium ADurb.BinA014, L-2,4-diaminobutyric acid acetyltransferase, 2.3.1.178, A0A1V5J550 | 28 |
| Bacillus halodurans, L-2,4-diaminobutyric acid acetyltransferase, 2.3.1.178, Q9KED3 | 29 |
| Bacillus sp. VT-16-64, L-ectoine synthase, 4.2.1.108, A0A1V2SL65 | 30 |
| Streptomyces anulatus, L-2,4-diaminobutyric acid acetyltransferase, 2.3.1.178, Q6QUZ0 | 31 |
| Tepidicaulis marinus, L-ectoine synthase, 4.2.1.108, A0A081BBU4 | 32 |
| Oceanobacillus iheyensis, L-2,4-diaminobutyric acid acetyltransferase, 2.3.1.178, Q8ESU9 | 33 |
| Leptospirillum ferriphilum, L-ectoine synthase, 4.2.1.108, J9Z7Y9 | 34 |
| Paenibacillus dendritiformis C454, L-2,4-diaminobutyric acid acetyltransferase, 2.3.1.178, H3SPK2 | 35 |
| Phenylobacterium sp. RIFCSPHIGHO2_01_FULL_70_10, L-ectoine synthase, 4.2.1.108, A0A1G2X194 | 36 |
| | DNA SEQ ID NO |
| Glaesserella parasuis, Diaminobutyrate--2-oxoglutarate aminotransferase, U4RVR1 | 37 |
| Glaesserella parasuis, Diaminobutyrate--2-oxoglutarate aminotransferase, U4RVR1 | 38 |
| Pseudomonas putida, Diaminobutyrate-2-oxoglutarate transaminase, Q88F75 | 39 |
| Methylomicrobium alcaliphilum, L-2,4-diaminobutyric acid acetyltransferase, Q4JQJ5 | 40 |
| Methylomicrobium alcaliphilum, L-2,4-diaminobutyric acid acetyltransferase, Q4JQJ5 | 41 |
| Desulfurispirillum indicum, L-2,4-diaminobutyric acid acetyltransferase, E6W3G0 | 42 |
| Desulfurispirillum indicum, L-2,4-diaminobutyric acid acetyltransferase, E6W3G0 | 43 |
| hydrothermal vent metagenome, L-ectoine synthase, A0A160TCS0 | 44 |
| hydrothermal vent metagenome, L-ectoine synthase, A0A160TCS0 | 45 |
| Alkalilimnicola ehrlichii, L-ectoine synthase, Q0A9E8 | 46 |
| Alkalilimnicola ehrlichii, L-ectoine synthase, Q0A9E8 | 47 |
| Methanobacterium formicicum, L-2,4-diaminobutyric acid acetyltransferase EctA, A0A09015X2 | 48 |
| Methanobacterium formicicum, L-2,4-diaminobutyric acid acetyltransferase EctA, A0A09015X2 | 49 |
| Hahella chejuensis, L-ectoine synthase, Q2SLV8 | 50 |
| Hahella chejuensis, L-ectoine synthase, Q2SLV8 | 51 |
| Methanosaeta sp. SDB, L-2,4-diaminobutyric acid acetyltransferase, A0A0Q1AWK1 | 52 |
| Methanosaeta sp. SDB, L-2,4-diaminobutyric acid acetyltransferase, A0A0Q1AWK1 | 53 |
| Phenylobacterium zucineum, L-ectoine synthase, B4R981 | 54 |
| Phenylobacterium zucineum, L-ectoine synthase, B4R981 | 55 |
| Methanothrix soehngenii, L-2,4-diaminobutyric acid acetyltransferase, F4C028 | 56 |
| Bacillus halodurans, L-ectoine synthase, Q9KED5 | 57 |
| Bacillus halodurans, L-ectoine synthase, Q9KED5 | 58 |
| Nitrosopumilus maritimus, L-2,4-diaminobutyric acid acetyltransferase, A9A2K3 | 59 |
| Nitrosopumilus maritimus, L-2,4-diaminobutyric acid acetyltransferase, A9A2K3 | 60 |
| Sphingopyxis alaskensis, L-ectoine synthase, Q1GNW6 | 61 |
| Sphingopyxis alaskensis, L-ectoine synthase, Q1GNW6 | 62 |
| Candidatus Entotheonella sp., Uncharacterized protein, W4LG62 | 63 |
| Candidatus Entotheonella sp., Uncharacterized protein, W4LG62 | 64 |
| Flexistipes sinusarabici, L-2,4-diaminobutyric acid acetyltransferase, F8E4K4 | 65 |
| Flexistipes sinusarabici, L-2,4-diaminobutyric acid acetyltransferase, F8E4K4 | 66 |
| Leptospirillum ferriphilum YSK, L-2,4-diaminobutyric acid acetyltransferase, A0A059XSI6 | 67 |
| Nitrincola nitratireducens, L-ectoine synthase, W9UWT5 | 68 |
| Nitrincola nitratireducens, L-ectoine synthase, W9UWT5 | 69 |
| Nitrospina sp. SCGC_AAA799_A02, L-2,4-diaminobutyric acid acetyltransferase, A0A0J6YS11 | 70 |
| Marinobacterium stanieri, L-ectoine synthase, A0A1N6PYR6 | 71 |
| Marinobacterium stanieri, L-ectoine synthase, A0A1N6PYR6 | 72 |
| Spirochaeta africana, L-2,4-diaminobutyric acid acetyltransferase, H9UMW7 | 73 |
| Actinomadura sp. CNU-125, L-ectoine synthase, A0A1Q9TDA5 | 74 |
| Actinomadura sp. CNU-125, L-ectoine synthase, A0A1Q9TDA5 | 75 |
| Dehalogenimonas lykanthroporepellens, L-2,4-diaminobutyric acid acetyltransferase, D8K1W8 | 76 |
| Dehalogenimonas lykanthroporepellens, L-2,4-diaminobutyric acid acetyltransferase, D8K1W8 | 77 |
| Leptospirillum ferriphilum YSK, L-ectoine synthase, A0A059XT79 | 78 |
| Leptospirillum ferriphilum YSK, L-ectoine synthase, A0A059XT79 | 79 |
| Bordetella bronchiseptica, L-2,4-diaminobutyric acid acetyltransferase, Q7WHI7 | 80 |
| Bordetella bronchiseptica, L-2,4-diaminobutyric acid acetyltransferase, Q7WHI7 | 81 |
| Betaproteobacteria bacterium SG8_41, L-ectoine synthase, A0A0S8C710 | 82 |
| Betaproteobacteria bacterium SG8_41, L-ectoine synthase, A0A0S8C710 | 83 |
| hydrothermal vent metagenome, L-ectoine synthase, A0A160TXJ7 | 84 |
| Deltaproteobacteria bacterium ADurb.BinA014, L-2,4-diaminobutyric acid acetyltransferase, A0A1V5J550 | 85 |
| Bacillus halodurans, L-2,4-diaminobutyric acid acetyltransferase, Q9KED3 | 86 |
| Bacillus halodurans, L-2,4-diaminobutyric acid acetyltransferase, Q9KED3 | 87 |
| Bacillus sp. VT-16-64, L-ectoine synthase, A0A1V2SL65 | 88 |
| Bacillus sp. VT-16-64, L-ectoine synthase, A0A1V2SL65 | 89 |
| Streptomyces anulatus, L-2,4-diaminobutyric acid acetyltransferase, Q6QUZ0 | 90 |
| Streptomyces anulatus, L-2,4-diaminobutyric acid acetyltransferase, Q6QUZ0 | 91 |
| Tepidicaulis marinus, L-ectoine synthase, A0A081BBU4 | 92 |
| Tepidicaulis marinus, L-ectoine synthase, A0A081BBU4 | 93 |
| Oceanobacillus iheyensis, L-2,4-diaminobutyric acid acetyltransferase, Q8ESU9 | 94 |
| Oceanobacillus iheyensis, L-2,4-diaminobutyric acid acetyltransferase, Q8ESU9 | 95 |
| Leptospirillum ferriphilum, L-ectoine synthase, J9Z7Y9 | 96 |
| Leptospirillum ferriphilum, L-ectoine synthase, J9Z7Y9 | 97 |
| Paenibacillus dendritiformis C454, L-2,4-diaminobutyric acid acetyltransferase, H3SPK2 | 98 |
| Phenylobacterium sp. RIFCSPHIGHO2_01_FULL_70_10, L-ectoine synthase, A0A1G2X194 | 99 |
| Phenylobacterium sp. RIFCSPHIGHO2_01_FULL_70_10, L-ectoine synthase, A0A1G2X194 | 100 |
| Corynebacterium glutamicum, promoter | 101 |

Microbial Host Cells

Any microbe that can be used to express introduced genes can be engineered for fermentative production of ectoine as described above. In certain embodiments, the microbe is one that is naturally incapable of fermentative production of ectoine. In some embodiments, the microbe is one that is readily cultured, such as, for example, a microbe known to be useful as a host cell in fermentative production of compounds of interest. Bacteria cells, including gram-positive or gram-negative bacteria can be engineered as described above. Examples include, in addition to C. glutamicum cells, Bacillus subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, P. citrea, Lactobacillus spp. (such as L. lactis, L. plantarum), L. grayi, E. coli, E. faecium, E. gallinarum, E. casseliflavus, and/or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances; (1989), 7(2):127-154). Examples include Trichoderma longibrachiatum, T. viride, T. koningii, T. harzianum, Penicillium sp., Humicola insolens, H. lanuginose, H. grisea, Chrysosporium sp., C. lucknowense, Gliocladium sp., Aspergillus sp. (such as A. oryzae, A. niger, A. sojae, A. japonicus. A. nidulans, or A. awamori), Fusarium sp. (such as F. roseum, F. graminum F. cerealis, F. oxysporum, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T. reesei, T. viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Patent Pub. No. 2011/0045563.

In some embodiments, the host cell can be an algal cell-derived, e.g., from a green alga, red alga, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Genetic Engineering Methods

Microbial cells can be engineered for fermentative ectoine production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, Apr. 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of C. glutamicum and S. cerevisiae cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

Engineered Microbial Cells

The above-described methods can be used to produce engineered microbial cells that produce, and in certain embodiments, overproduce, ectoine. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Examples below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for ectoine production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least three heterologous genes, e.g., a non-native diaminobutyrate aminotransferase, a non-native L-2,4-diaminobutyric acid transaminase, and a non-native ectoine synthase gene. In various embodiments, the microbial cell can include and express, for example: (1) a single copy of each of these genes, (2) two or more copies of one of these genes, which can be the same or different, or (3) two or more copies of two or all three of these genes, wherein the copies of a given gene can be the same or different. The same is true for other heterologous genes that can be introduced into the engineered microbial cell.

This engineered host cell can include at least one additional genetic alteration that increases flux through any pathway leading to the production of an immediate precursor of diaminobutyric acid (e.g., aspartate semi-aldehyde). As discussed above, this can be accomplished, for example, by increasing the activity of one or more upstream enzymes.

The engineered microbial cells can contain introduced genes that have a native nucleotide sequence or that differ from native. For example, the native (or non-native) nucleotide sequence can be codon-optimized for expression in a particular host cell. Codon optimization for a particular host can, for example, be based on the codon usage tables found at www.kazusa.or.jp/codon/. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

The approach described herein has been carried out in bacterial cells, namely C. glutamicum. (See Examples 1-4.)

Illustrative Engineered Bacterial Cells

Production of Diaminobutyric Acid

In certain embodiments, the engineered bacterial (e.g., C. glutamicum) cell expresses one or more non-native diaminobutyrate aminotransferase(s) having at least 70 percent, 75 percent, 80 percent, 85 percent, 90 percent, 95 percent or 100 percent amino acid sequence identity with a diaminobutyrate aminotransferase encoded by an Glaesserella parasuis MN-H diaminobutyrate aminotransferase gene (e.g., SEQ ID NO:1) and/or one or more non-native diaminobutyrate aminotransferase(s) having at least 70 percent, 75 percent, percent, 85 percent, 90 percent, 95 percent or 100 percent amino acid sequence identity with a diaminobutyrate aminotransferase encoded by a Pseudomonas putida diaminobutyrate aminotransferase gene (e.g., SEQ ID NO:2).

In particular embodiments:
the non-native Glaesserella parasuis MN-H diaminobutyrate aminotransferase includes SEQ ID NO:1; and
the non-native Pseudomonas putida diaminobutyrate aminotransferase includes SEQ ID NO:2.

In C. glutamicum, for example, titers of diaminobutyric acid exceeding 900 mg/L were achieved by overexpressing (by expressing two copies of the genes) the enzymes having SEQ ID NOs:1 and 2 (see Example 3).

Production of Ectoine

In certain embodiments, the engineered bacterial (e.g., C. glutamicum) cell expresses one or more non-native diaminobutyrate aminotransferase(s) having at least 70 percent, 75 percent, 80 percent, 85 percent, 90 percent, 95 percent or 100 percent amino acid sequence identity with a diaminobutyrate aminotransferase encoded by a Pseudomonas putida diaminobutyrate aminotransferase gene (e.g., SEQ ID NO:2) to enable production of diaminobutyric acid.

To enable production of ectoine, this engineered bacterial cell additionally expresses one or more non-native L-2,4-diaminobutyrate acetyltransferase(s) having at least percent, 75 percent, 80 percent. 85 percent, 90 percent, 95 percent or 100 percent amino acid sequence identity with an L-2,4-diaminobutyrate acetyltransferase encoded by a Methylomicrobium alcaliphilum (strain DSM 19304/NCIMB 14124/VKM B-2133/20Z) L-2,4-diaminobutyrate acetyltransferase gene (e.g., SEQ ID NO:3) and one or more non-native ectoine synthase(s) encoded by a hydrothermal vent metagenome ectione synthase gene (e.g., SEQ ID NO:5).

In particular embodiments:
the non-native Methylomicrobium alcaliphilum (strain DSM 19304/NCIMB 14124/VKM B-2133/20Z) L-2,4-diaminobutyrate acetyltransferase includes SEQ ID NO:3; and
the non-native hydrothermal vent metagenome ectione synthase includes SEQ ID NO:5.

In C. glutamicum, for example, an ectoine titer of about 530 mg/L were achieved by expressing the enzymes having SEQ ID NOs:2, 3 and 5 (see Example 4).

Culturing of Engineered Microbial Cells

Any of the microbial cells described herein can be cultured, e.g., for maintenance, growth, and/or ectoine production.

In some embodiments, the cultures are grown to an optical density at 600 nm of 10-500, such as an optical density of 50-150.

In various embodiments, the cultures include produced ectoine at titers of at least 100, 200, 300, 400, 500, 600, 700, 800, or 900 mg/L or at least 1, 1.5, 2, 2.5, 3, 3.5, 4, 4.5, 5, 10, 15, 20, or 25 gm/L. In various embodiments, the titer is in the range of 100 mg/L to 25 gm/L, 300 mg/L to 10 gm/L, 500 mg/L to 5 gm/L, 600 mg/L to 4 gm/L, 700 mg/L to 3 gm/L, 800 mg/L to 2 gm/L, 900 mg/L to 1.5 gm/L or any range bounded by any of the values listed above.

Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce ectoine, the culture medium can include, and/or is supplemented during culture with, glucose and/or a nitrogen source such as urea, an ammonium salt, ammonia, or any combination thereof.

Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO2, and a pH between about 5 to about 9). In some aspects, cells are grown at 35° C. In certain embodiments, such as where thermophilic bacteria are used as the host cells, higher temperatures (e.g., 50° C.-75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), (w/v), 0.2% (w/v), or OA % (w/v) glucose. In some cultures, significantly higher levels of sugar (e.g., glucose) are used, e.g., at least 10% (w/v), 20% (w/v), 30% (w/v), % (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70% (w/v), 20-60% (w/v), or 30-% (w/v). Furthermore, different sugar levels can be used for different phases of culturing. For fed-batch culture (e.g., of S. cerevisiae or C. glutamicum), the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

### Ectoine Production and Recovery

Any of the methods described herein may further include a step of recovering ectoine. In some embodiments, the produced ectoine contained in a so-called harvest stream is recovered/harvested from the production vessel. The harvest stream may include, for instance, cell-free or cell-containing aqueous solution coming from the production vessel, which contains ectoine as a result of the conversion of production substrate by the resting cells in the production vessel. Cells still present in the harvest stream may be separated from the ectoine by any operations known in the art, such as for instance filtration, centrifugation, decantation, membrane crossflow ultrafiltration or microfiltration, tangential flow ultrafiltration or microfiltration or dead-end filtration. After this cell separation operation, the harvest stream is essentially free of cells.

Further steps of separation and/or purification of the produced ectoine from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify ectoine. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### Example 1—First-Round Genetic Engineering of Corynebacterium glutamicum to Produce Diaminobutyric Acid

Plasmid/DNA Design

All strains tested for this work were transformed with plasmid DNA designed using proprietary software. Plasmid designs were specific to each of the host organisms engineered in this work. The plasmid DNA was physically constructed by a standard DNA assembly method. This plasmid DNA was then used to integrate metabolic pathway inserts by one of two host-specific methods, each described below.

C. glutamicum Pathway Integration

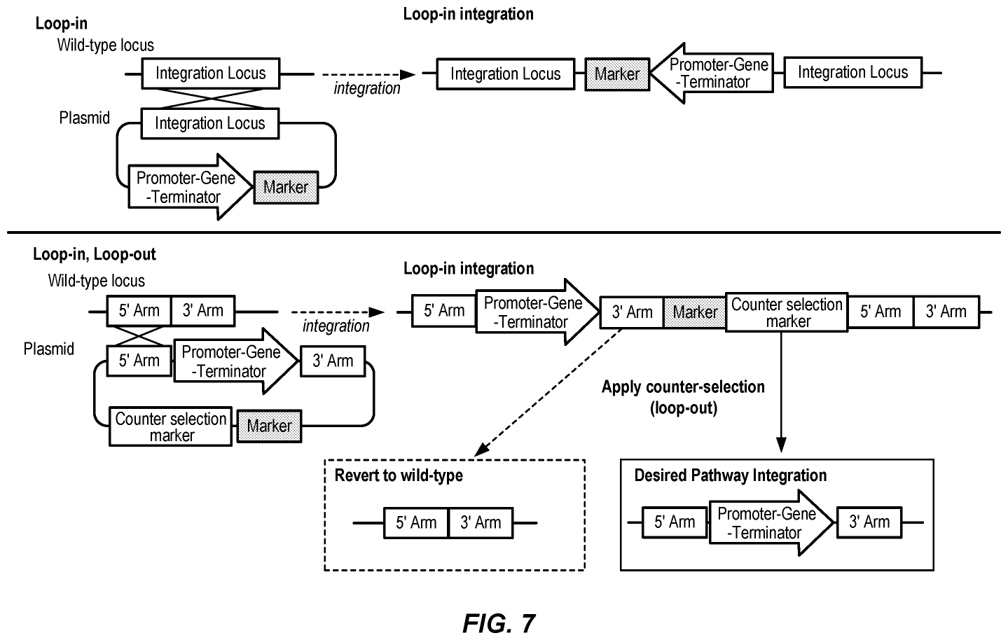

A “loop-in, single-crossover” genomic integration strategy has been developed to engineer C. glutamicum strains. FIG. 7 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum chromosome. Integration events are stably maintained in the genome by growth in the presence of antibiotic (25 μg/ml kanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF=upstream forward, DR=downstream reverse, IR=internal reverse, IF=internal forward.)

S. cerevisiae Pathway Integration

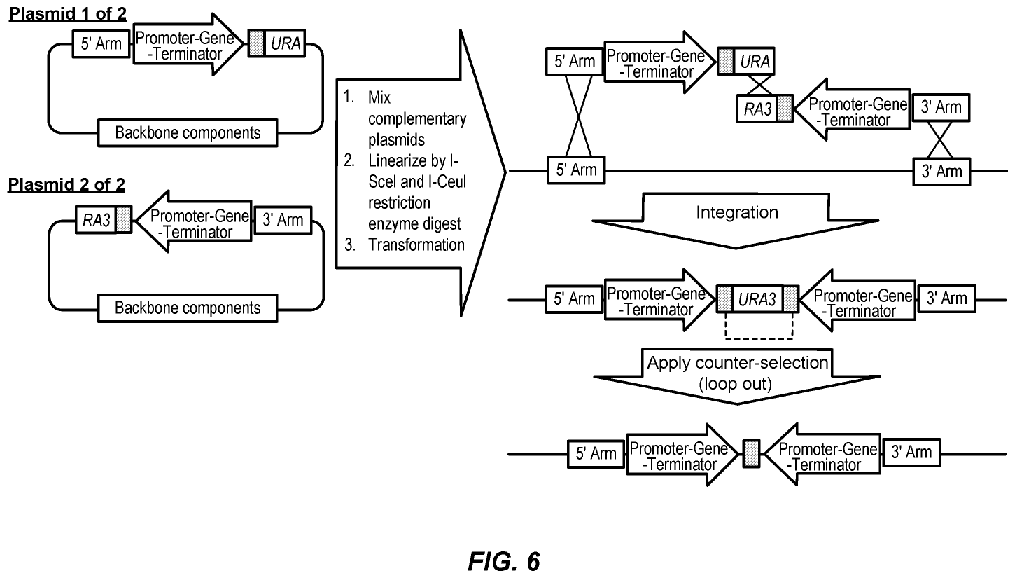

A “split-marker, double-crossover” genomic integration strategy has been developed to engineer S. cerevisiae strains. FIG. 6 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knock-out, gene knock-in, and promoter titration in the same workflow.

Cell Culture

The workflow established for C. glutamicum involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (BHI for C. glutamicum, SD-ura) and cultivated for two days until saturation and then frozen with 16.6% glycerol at −80° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 754 of supernatant was transferred to each plate, with one stored at 4° C., and the second stored at 80° C. for long-term storage.

First-Round Genetic Engineering Results

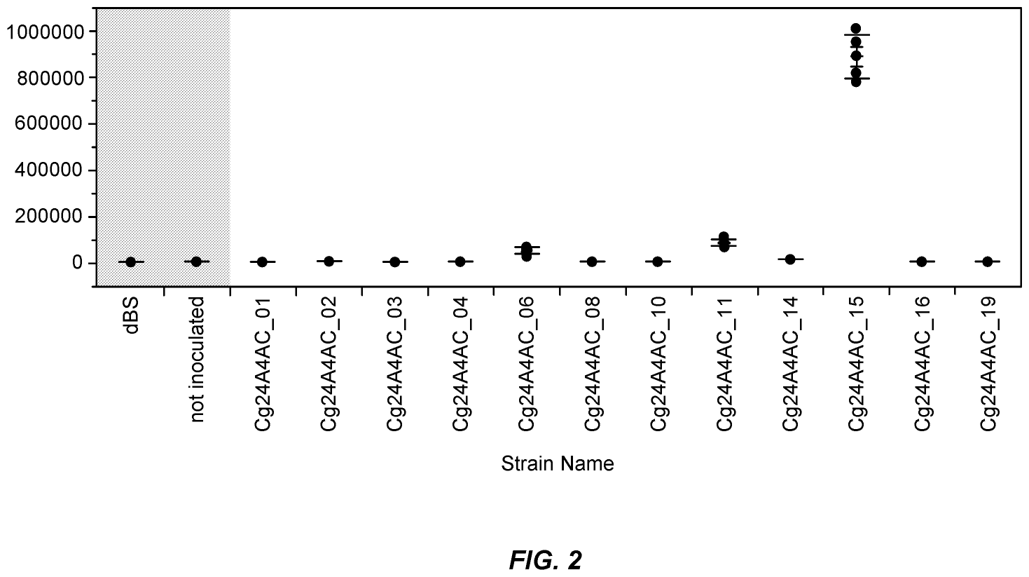

This cycle of genetic engineering was aimed at producing the ectoine precursor diaminobutyric acid. During this cycle, a library of single heterologous gene, diaminobutyrate aminotransferase (diaminobutyrate aminotransferase), was inserted. As shown in FIG. 2, while a large number of strains (19 out of the 20 attempted) were generated, the titer difference between the winning strain and the next-best was over 10-fold. The enzyme giving the highest titer was the diaminobutyrate aminotransferase (diaminobutyrate synthase) from Glaesserella parasuis MN-H (Uniprot ID U4RVR1), yielding a titer of ˜900 mg/L (Cg24A4AC_15).

### Example 2—Second-Round Genetic Engineering of Corynebacterium glutamicum to Produce Diaminobutyric Acid

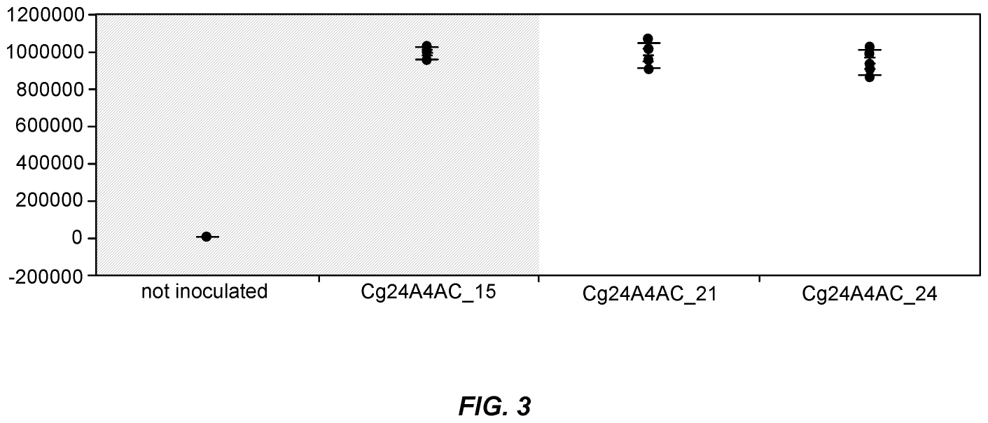

During the second cycle of genetic engineering toward diaminobutyric acid production, promoter swaps (proswaps) were used to upregulate particular genes that encode enzymes in the pathway leading to aspartate semi-aldehyde. The approach consisted of incorporating pre-constructed factory-built proswaps into the previous cycle winner (Cg24A4AC_15). At the completion of the cycle, only two strains were successfully constructed; inspection of the strains (Cg24A4AC_21 and Cg24A4AC_24) indicates that these strains are identical. Both consist of a strongly upregulated promoter on aspartokinase (NCg10247 with Pcg0007_39). The results, shown in FIG. 3, indicate a negligible effect on diaminobutyric acid titer.

### Example 3—Third-Round Genetic Engineering of Corynebacterium glutamicum to Produce Diaminobutyric Acid

The approach to the third cycle of genetic engineering toward diaminobutyric acid production included overexpression of enzymes catalyzing pathway steps leading to diaminobutyric acid. Multiple versions of enzymatic steps were explored. These enzymes consisted of:
aspartate transaminase;
aspartate kinase;
aspartate semialdehyde dehydrogenase;
diaminobutyrate aminotransferase; and
malate dehydrogenase.

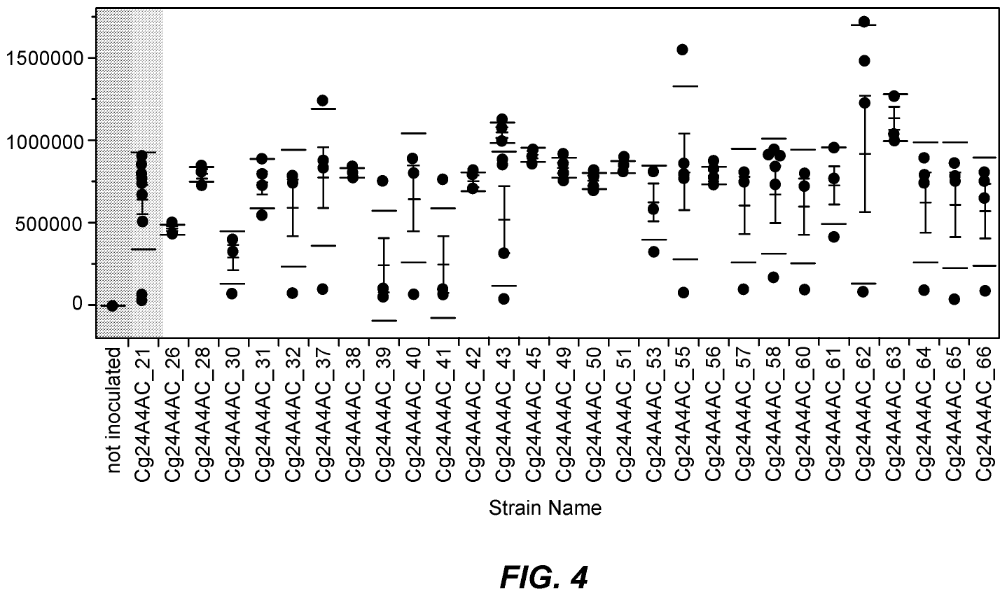

Round-to-round variations in overall titers resulted in a drop in titer for the control strain in this cycle from ˜999 mg/L to ˜650 mg/L. Additionally, a number of nearly zero-titer wells skew titer values downward. Two strains (Cg2A4AC_62 and Cg2A4AC_63) outperform the remaining strains. These strains are single overexpressing strains containing an additional copy of the heterologous step, diaminobutyrate aminotransferase. One of the top-performing strains (Cg2A4AC_62) contains two copies of the gene encoding the winning enzyme from the first DBTA cycle, diaminobutyrate aminotransferase from Glaesserella parasuis MN-H (Uniprot ID U4RVR1), while Cg2A4AC_63 contains one copy of this gene and one copy of a gene encoding a previously untested enzyme (from Pseudomonas putida with Uniprot ID Q88F75). A diaminobutyric acid titer of ˜1.4 g/L was obtained (see FIG. 4).

### Example 4—Engineering of Corynebacterium glutamicum to Produce Ectoine

A single round of engineering of Corynebacterium glutamicum to produce ectoine was carried out. Further genetic alterations were built into the best strain from Example 3 (Cg24A4AC_63). In particular, two additional genes needed to produce L-ectione from diaminobutyric acid, L-2,4-diaminobutyric acid transaminase, and ectoine synthase, were inserted into Cg24A4AC_63. This round of strain engineering resulted in ectoine titers of up to 530 mg/L in C. glutamicum. The best-producing strain (CgECTOIN_13) included a Methylomicrobium alcaliphilum (strain DSM 19304/NCIMB 14124/VKM B-2133/20Z) L-2,4-diaminobutyrate acetyltransferase (SEQ ID NO:3) and a non-native hydrothermal vent metagenome L-ectione synthase (SEQ ID NO:5).

### Genetic Engineering Results Tables

TABLE 1
Genetic Engineering of Corynebacterium glutamicum to Produce Diaminobutyric Acid
Strain Cg24A4AC_15 was used as the parent strain for all cycle 2 strains; therefore, cycle 2 strains contained diaminobutyrate
aminotransferase from Glaesserella parasuis MN-H (Uniprot ID U4RVR1), in addition to the genetic alterations shown in Table 1.
Strain Cg24A4AC_21 was used as the parent strain for all cycle 3 strains; therefore cycle 3 strains contained diaminobutyrate
aminotransferase from Glaesserella parasuis MN-H (Uniprot ID U4RVR1) and a strongly upregulated promoter on
aspartokinase (NCgl0247 with Pcg0007_39), in addition to the genetic alterations shown in Table 1. (No data were obtained
for strain designs where no titer is indicated.)

| | | | | | | Enzyme | | | Enzyme | | |
|---|---|---|---|---|---|---|---|---|---|---|---|
| | | | Enzyme 1- | E1 | Enzyme 1- | 2- | E2 | Enzyme 2- | 3- | E3 | Enzyme 3- |
| Strn. | | | activity | Uniprot | source | activity | Uniprot | source | activity | Uniprot | source |
| name | Cycle | Titer (μg/L) | name | ID | organism | name | ID | organism | name | ID | organism |
| Cg24 | 1 | 28825.374 | diamino- | D4 | Haloferax | | | | | | |
| A4A | | | butyrate | GP | volcanii | | | | | | |
| C_01 | | | amino- | 48 | (strain | | | | | | |
| | | | transferase | | ATCC | | | | | | |
| | | | | | 29605/ | | | | | | |
| | | | | | DSM 3757/ | | | | | | |
| | | | | | JCM 8879/ | | | | | | |
| | | | | | NBRC | | | | | | |
| | | | | | 14742/ | | | | | | |
| | | | | | NCIMB | | | | | | |
| | | | | | 2012/ | | | | | | |
| | | | | | VKM | | | | | | |
| | | | | | B-1768/ | | | | | | |
| | | | | | DS2) | | | | | | |
| | | | | | (Halo- | | | | | | |
| | | | | | bacterium | | | | | | |
| | | | | | volcanii) | | | | | | |
| Cg24 | 1 | 21045.919 | diamino- | G0 | Haloarcula | | | | | | |
| A4A | | | butyrate | HY | hispanica | | | | | | |
| C_02 | | | amino- | R4 | (strain | | | | | | |
| | | | transferase | | ATCC | | | | | | |
| | | | | | 33960/ | | | | | | |
| | | | | | DSM 4426/ | | | | | | |
| | | | | | JCM 8911/ | | | | | | |
| | | | | | NBRC | | | | | | |
| | | | | | 102182/ | | | | | | |
| | | | | | NCIMB | | | | | | |
| | | | | | 2187/ | | | | | | |
| | | | | | VKM | | | | | | |
| | | | | | B-1755) | | | | | | |
| Cg24 | 1 | 19765.801 | diamino- | A0A | Marine | | | | | | |
| A4A | | | butyrate | 081 | Group I | | | | | | |
| C_03 | | | amino- | RM | thaumar- | | | | | | |
| | | | transferase | V4 | chaeote | | | | | | |
| | | | | | SCGC | | | | | | |
| | | | | | AAA799- | | | | | | |
| | | | | | N04 | | | | | | |
| Cg24 | 1 | 33528.232 | diamino- | B0 | Halo- | | | | | | |
| A4A | | | butyrate | R9 | bacterium | | | | | | |
| C_04 | | | amino- | P8 | salinarum | | | | | | |
| | | | transferase | | (strain | | | | | | |
| | | | | | ATCC | | | | | | |
| | | | | | 29341/ | | | | | | |
| | | | | | DSM | | | | | | |
| | | | | | 671/R1) | | | | | | |
| Cg24 | 1 | | diamino- | Q93 | Strepto- | | | | | | |
| A4A | | | butyrate | RW | myces | | | | | | |
| C_05 | | | amino- | 1 | coelicolor | | | | | | |
| | | | transferase | | (strain | | | | | | |
| | | | | | ATCC | | | | | | |
| | | | | | BAA- | | | | | | |
| | | | | | 471/ | | | | | | |
| | | | | | A3(2)/ | | | | | | |
| | | | | | M145) | | | | | | |
| Cg24 | 1 | 40553.205 | diamino- | J2J | Strepto- | | | | | | |
| A4A | | | butyrate | VG | myces | | | | | | |
| C_06 | | | amino- | 2 | auratus | | | | | | |
| | | | transferase | | AGR0001 | | | | | | |
| Cg24 | 1 | | diamino- | Q6 | Strepto- | | | | | | |
| A4A | | | butyrate | QU | myces | | | | | | |
| C_07 | | | amino- | Y9 | anulatus | | | | | | |
| | | | transferase | | (Strepto- | | | | | | |
| | | | | | myces | | | | | | |
| | | | | | chrysomallus) | | | | | | |
| Cg24 | 1 | 29531.977 | diamino- | A0A | Myco- | | | | | | |
| A4A | | | butyrate | 0U1 | bacterium | | | | | | |
| C_08 | | | amino- | BE | abscessus | | | | | | |
| | | | transferase | H0 | | | | | | | |
| Cg24 | 1 | | diamino- | X8E | Myco- | | | | | | |
| A4A | | | butyrate | V65 | bacterium | | | | | | |
| C_09 | | | amino- | | chelonae | | | | | | |
| | | | transferase | | 1518 | | | | | | |
| Cg24 | 1 | 53403.275 | diamino- | A0A | Strepto- | | | | | | |
| A4A | | | butyrate | 124 | myces | | | | | | |
| C_10 | | | amino- | C5 | scabiei | | | | | | |
| | | | transferase | M5 | | | | | | | |
| Cg24 | 1 | 54323.134 | diamino- | A0A | Strepto- | | | | | | |
| A4A | | | butyrate | 087 | myces | | | | | | |
| C_11 | | | amino- | KB0 | sp. JS01 | | | | | | |
| | | | transferase | 8 | | | | | | | |
| Cg24 | 1 | | diamino- | A0A | Strepto- | | | | | | |
| A4A | | | butyrate | 0B5 | myces | | | | | | |
| C_12 | | | amino- | HN | vietnam- | | | | | | |
| | | | transferase | 20 | ensis | | | | | | |
| Cg24 | 1 | | diamino- | O52 | Halomonas | | | | | | |
| 44A | | | butyrate | 250 | elongata | | | | | | |
| C_13 | | | amino- | | (strain | | | | | | |
| | | | transferase | | ATCC | | | | | | |
| | | | | | 33173/ | | | | | | |
| | | | | | DSM 2581/ | | | | | | |
| | | | | | NBRC | | | | | | |
| | | | | | 15536/ | | | | | | |
| | | | | | NCIMB | | | | | | |
| | | | | | 2198/ | | | | | | |
| | | | | | 1H9) | | | | | | |
| Cg24 | 1 | 528005.35 | diamino- | U4 | Glaesserella | | | | | | |
| A4A | | | butyrate | RV | parasuis | | | | | | |
| C_15 | | | amino- | R1 | MN-H | | | | | | |
| | | | transferase | | | | | | | | |
| Cg24 | 1 | 9942.4326 | diamino- | P56 | Acinetobacter | | | | | | |
| A4A | | | butyrate | 744 | baumannii | | | | | | |
| C_16 | | | amino- | | | | | | | | |
| | | | transferase | | | | | | | | |
| Cg24 | 1 | | diamino- | A0A | Photo- | | | | | | |
| A4A | | | butyrate | 090 | bacterium | | | | | | |
| C_17 | | | amino- | R3 | aphoticum | | | | | | |
| | | | transferase | G5 | | | | | | | |
| Cg24 | 1 | | diamino- | Q88 | Pseudomonas | | | | | | |
| A4A | | | butyrate | F75 | putida (strain | | | | | | |
| C_18 | | | amino- | | KT2440) | | | | | | |
| | | | transferase | | | | | | | | |
| Cg24 | 1 | 13552.643 | diamino- | Q9 | Rhizobium | | | | | | |
| A4A | | | butyrate | Z3R | meliloti | | | | | | |
| C_19 | | | amino- | 2 | (strain | | | | | | |
| | | | transferase | | 1021) | | | | | | |
| | | | | | (Ensifer | | | | | | |
| | | | | | meliloti) | | | | | | |
| | | | | | (Sinorhizo- | | | | | | |
| | | | | | bium | | | | | | |
| | | | | | meliloti) | | | | | | |
| Cg24 | 2 | 410871.3 | native | | | | | | | | |
| A4A | | | asparto- | | | | | | | | |
| C_21 | | | kinase | | | | | | | | |
| | | | upregulated | | | | | | | | |
| | | | by promoter | | | | | | | | |
| | | | swap | | | | | | | | |
| Cg24 | 2 | 494780.32 | native | | | | | | | | |
| A4A | | | asparto- | | | | | | | | |
| C_24 | | | kinase | | | | | | | | |
| | | | upregulated | | | | | | | | |
| | | | by promoter | | | | | | | | |
| | | | swap | | | | | | | | |
| Cg24 | 3 | 207439.02 | Asparto- | P00 | Escherichia | | | | | | |
| A4A | | | kinase I/ | 561 | coli | | | | | | |
| C_26 | | | Homoserine | | (strain K12) | | | | | | |
| | | | dehydro- | | | | | | | | |
| | | | genase I | | | | | | | | |
| Cg24 | 3 | | Aspartate | P10 | Saccharo- | | | | | | |
| A4A | | | kinase | 869 | myces | | | | | | |
| C_27 | | | | | cerevisiae | | | | | | |
| | | | | | (strain | | | | | | |
| | | | | | ATCC | | | | | | |
| | | | | | 204508/ | | | | | | |
| | | | | | S288c) | | | | | | |
| | | | | | (Baker's | | | | | | |
| | | | | | yeast) | | | | | | |
| Cg24 | 3 | 326642.41 | aspartate | P26 | Coryne- | | | | | | |
| A4A | | | kinase | 512 | bacterium | | | | | | |
| C_28 | | | activity | | glutamicum | | | | | | |
| Cg24 | 3 | | Asparto- | P08 | Escherichia | | | | | | |
| A4A | | | kinase III | 660 | coli | | | | | | |
| C_29 | | | | | (strain K12) | | | | | | |
| Cg24 | 3 | 223269.3 | Asparto- | P08 | Escherichia | | | | | | |
| A4A | | | kinase III | 660 | coli | | | | | | |
| C_30 | | | | | (strain K12) | | | | | | |
| Cg24 | 3 | 295703.78 | Asparto- | P08 | Escherichia | | | | | | |
| A4A | | | kinase III | 660 | coli | | | | | | |
| C_31 | | | | | (strain K12) | | | | | | |
| Cg24 | 3 | 344765.62 | Asparto- | P08 | Escherichia | | | | | | |
| A4A | | | kinase III | 660 | coli | | | | | | |
| C_32 | | | | | (strain K12) | | | | | | |
| Cg24 | 3 | | Asparto- | P08 | Escherichia | | | | | | |
| A4A | | | kinase III | 660 | coli | | | | | | |
| C_33 | | | | | (strain K12) | | | | | | |
| Cg24 | 3 | | Asparto- | P08 | Escherichia | | | | | | |
| A4A | | | kinase III | 660 | coli | | | | | | |
| C_34 | | | | | (strain K12) | | | | | | |
| Cg24 | 3 | | Asparto- | P08 | Escherichia | | | | | | |
| A4A | | | kinase III | 660 | coli | | | | | | |
| C_35 | | | | | (strain K12) | | | | | | |
| Cg24 | 3 | | Asparto- | P26 | Coryne- | | | | | | |
| A4A | | | kinase | 512 | bacterium | | | | | | |
| C_36 | | | | | glutamicum | | | | | | |
| | | | | | (strain | | | | | | |
| | | | | | ATCC | | | | | | |
| | | | | | 13032/DSM | | | | | | |
| | | | | | 20300/JCM | | | | | | |
| | | | | | 1318/LMG | | | | | | |
| | | | | | 3730/ | | | | | | |
| | | | | | NCIMB | | | | | | |
| | | | | | 10025) | | | | | | |
| Cg24 | 3 | 519333.11 | Asparto- | P26 | Coryne- | | | | | | |
| A4A | | | kinase | 512 | bacterium | | | | | | |
| C_37 | | | | | glutamicum | | | | | | |
| | | | | | (strain | | | | | | |
| | | | | | ATCC | | | | | | |
| | | | | | 13032/DSM | | | | | | |
| | | | | | 20300/JCM | | | | | | |
| | | | | | 1318/LMG | | | | | | |
| | | | | | 3730/ | | | | | | |
| | | | | | NCIMB | | | | | | |
| | | | | | 10025) | | | | | | |
| Cg24 | 3 | 460685.23 | Aspartate- | P32 | Saccharo- | Malate | Q12 | Saccharo- | | | |
| A4A | | | semi- | 801 | myces | dehydro- | 128 | myces | | | |
| C_38 | | | aldehyde | | cerevisiae | genase | | cerevisiae | | | |
| | | | dehydro- | | (strain | | | (strain | | | |
| | | | genase | | ATCC | | | ATCC | | | |
| | | | (ASA | | 204508/ | | | 204508/ | | | |
| | | | dehydro- | | S288c) | | | S288c) | | | |
| | | | genase) | | (Baker's | | | (Baker's | | | |
| | | | (ASADH) | | yeast) | | | yeast) | | | |
| Cg24 | 3 | 272216.14 | Aspartate- | P0 | Coryne- | Aspartate | Q8 | Coryne- | | | |
| A4A | | | semi- | C1 | bacterium | trans- | NT | bacterium | | | |
| C_39 | | | aldehyde | D8 | glutamicum | aminase | R2 | glutamicum | | | |
| | | | dehydro- | | (strain | | | (strain | | | |
| | | | genase | | ATCC | | | ATCC | | | |
| | | | (ASA | | 13032/ | | | 13032/ | | | |
| | | | dehydro- | | DSM | | | DSM | | | |
| | | | genase) | | 20300/ | | | 20300/ | | | |
| | | | (ASADH) | | JCM | | | JCM | | | |
| | | | | | 1318/LMG | | | 1318/LMG | | | |
| | | | | | 3730/ | | | 3730/ | | | |
| | | | | | NCIMB | | | NCIMB | | | |
| | | | | | 10025) | | | 10025) | | | |
| Cg24 | 3 | 480879.55 | aspartate | P0 | Coryne- | Aspartate | Q8 | Coryne- | | | |
| A4A | | | semi- | C1 | bacterium | trans- | NT | bacterium | | | |
| C_40 | | | aldehyde | D8 | glutamicum | aminase | R2 | glutamicum | | | |
| | | | dehydro- | | (strain | | | (strain | | | |
| | | | genase | | ATCC | | | ATCC | | | |
| | | | | | 13032/ | | | 13032/ | | | |
| | | | | | DSM | | | DSM | | | |
| | | | | | 20300/ | | | 20300/ | | | |
| | | | | | JCM | | | JCM | | | |
| | | | | | 1318/LMG | | | 1318/LMG | | | |
| | | | | | 3730/ | | | 3730/ | | | |
| | | | | | NCIMB | | | NCIMB | | | |
| | | | | | 10025) | | | 10025) | | | |
| Cg24 | 3 | 467359.46 | Aspartate- | P0 | Coryne- | Malate | Q8 | Coryne- | | | |
| A4A | | | semi- | C1 | bacterium | dehydro- | NN | bacterium | | | |
| C_41 | | | aldehyde | D8 | glutamicum | genase | 33 | glutamicum | | | |
| | | | dehydro- | | (strain | | | (strain | | | |
| | | | genase | | ATCC | | | ATCC | | | |
| | | | (ASA | | 13032/ | | | 13032/ | | | |
| | | | dehydro- | | DSM | | | DSM | | | |
| | | | genase) | | 20300/JCM | | | 20300/JCM | | | |
| | | | (ASADH) | | 1318/LMG | | | 1318/LMG | | | |
| | | | | | 3730/ | | | 3730/ | | | |
| | | | | | NCIMB | | | NCIMB | | | |
| | | | | | 10025) | | | 10025) | | | |
| Cg24 | 3 | 392273.58 | Aspartate- | P32 | Saccharo- | aspartate | P23 | Saccharo- | | | |
| A4A | | | semi- | 801 | myces | amino- | 542 | myces | | | |
| C_42 | | | aldehyde | | cerevisiae | transferase | | cerevisiae | | | |
| | | | dehydro- | | (strain | activity | | (strain | | | |
| | | | genase | | ATCC | | | ATCC | | | |
| | | | (ASA | | 204508/ | | | 204508/ | | | |
| | | | dehydro- | | S288c) | | | S288c) | | | |
| | | | genase) | | (Baker's | | | (Baker's | | | |
| | | | (ASADH) | | yeast) | | | yeast) | | | |
| Cg24 | 3 | 418504.41 | aspartate | P0 | Coryne- | Malate | Q8 | Coryne- | | | |
| A4A | | | semi- | C1 | bacterium | dehydro- | NN | bacterium | | | |
| C_43 | | | aldehyde | D8 | glutamicum | genase | 33 | glutamicum | | | |
| | | | dehydro- | | (strain | | | (strain | | | |
| | | | genase | | ATCC | | | ATCC | | | |
| | | | | | 13032/ | | | 13032/ | | | |
| | | | | | DSM | | | DSM | | | |
| | | | | | 20300/JCM | | | 20300/JCM | | | |
| | | | | | 1318/LMG | | | 1318/LMG | | | |
| | | | | | 3730/ | | | 3730/ | | | |
| | | | | | NCIMB | | | NCIMB | | | |
| | | | | | 10025) | | | 10025) | | | |
| Cg24 | 3 | | aspartate | P0 | Coryne- | Aspartate | Q8 | Coryne- | aspartate | P26 | Coryne- |
| A4A | | | semi- | C1 | bacterium | trans- | NT | bacterium | kinase | 512 | bacterium |
| C_44 | | | aldehyde | D8 | glutamicum | aminase | R2 | glutamicum | activity | | glutamicum |
| | | | dehydro- | | (strain | | | (strain | | | |
| | | | genase | | ATCC | | | ATCC | | | |
| | | | | | 13032/ | | | 13032/ | | | |
| | | | | | DSM | | | DSM | | | |
| | | | | | 20300/JCM | | | 20300/JCM | | | |
| | | | | | 1318/LMG | | | 1318/LMG | | | |
| | | | | | 3730/ | | | 3730/ | | | |
| | | | | | NCIMB | | | NCIMB | | | |
| | | | | | 10025) | | | 10025) | | | |
| Cg24 | 3 | 533856.09 | Aspartate- | P32 | Saccharo- | aspartate | P23 | Saccharo- | Malate | Q12 | Saccharo- |
| A4A | | | semi- | 801 | myces | amino- | 542 | myces | dehydro- | 128 | myces |
| C_45 | | | aldehyde | | cerevisiae | transferase | | cerevisiae | genase | | cerevisiae |
| | | | dehydro- | | (strain | activity | | (strain | | | (strain |
| | | | genase | | ATCC | | | ATCC | | | ATCC |
| | | | (ASA | | 204508/ | | | 204508/ | | | 204508/ |
| | | | dehydro- | | S288c) | | | S288c) | | | S288c) |
| | | | genase) | | (Baker's | | | (Baker's | | | (Baker's |
| | | | (ASADH) | | yeast) | | | yeast) | | | yeast) |
| Cg24 | 3 | | aspartate | P0 | Coryne- | Malate | Q8 | Coryne- | Aspartate | Q8 | Coryne- |
| A4A | | | semi- | C1 | bacterium | dehydro- | NN | bacterium | trans- | NT | bacterium |
| C_46 | | | aldehyde | D8 | glutamicum | genase | 33 | glutamicum | aminase | R2 | glutamicum |
| | | | dehydro- | | (strain | | | (strain | | | (strain |
| | | | genase | | ATCC | | | ATCC | | | ATCC |
| | | | | | 13032/ | | | 13032/ | | | 13032/ |
| | | | | | DSM | | | DSM | | | DSM |
| | | | | | 20300/JCM | | | 20300/JCM | | | 20300/JCM |
| | | | | | 1318/LMG | | | 1318/LMG | | | 1318/LMG |
| | | | | | 3730/ | | | 3730/ | | | 3730/ |
| | | | | | NCIMB | | | NCIMB | | | NCIMB |
| | | | | | 10025) | | | 10025) | | | 10025) |
| Cg24 | 3 | | Aspartate | Q8 | Coryne- | | | | | | |
| A4A | | | trans- | NT | bacterium | | | | | | |
| C_47 | | | aminase | R2 | glutamicum | | | | | | |
| | | | | | (strain | | | | | | |
| | | | | | ATCC | | | | | | |
| | | | | | 13032/ | | | | | | |
| | | | | | DSM | | | | | | |
| | | | | | 20300/JCM | | | | | | |
| | | | | | 1318/LMG | | | | | | |
| | | | | | 3730/ | | | | | | |
| | | | | | NCIMB | | | | | | |
| | | | | | 10025) | | | | | | |
| Cg24 | 3 | | aspartate | P0 | Coryne- | | | | | | |
| A4A | | | semi- | C1 | bacterium | | | | | | |
| C_48 | | | aldehyde | D8 | glutamicum | | | | | | |
| | | | dehydro- | | (strain | | | | | | |
| | | | genase | | ATCC | | | | | | |
| | | | | | 13032/ | | | | | | |
| | | | | | DSM | | | | | | |
| | | | | | 20300/JCM | | | | | | |
| | | | | | 1318/LMG | | | | | | |
| | | | | | 3730/ | | | | | | |
| | | | | | NCIMB | | | | | | |
| | | | | | 10025) | | | | | | |
| Cg24 | | 419429.25 | diamino- | A0A | Strepto- | | | | | | |
| A4A | | | butyrate | 124 | myces | | | | | | |
| C_49 | | | amino- | C5 | scabiei | | | | | | |
| | | | transferase | M5 | | | | | | | |
| Cg24 | | 394573.75 | Aspartate | Q01 | Saccharo- | | | | | | |
| A4A | | | trans- | 802 | myces | | | | | | |
| C_50 | | | aminase | | cerevisiae | | | | | | |
| | | | | | (strain | | | | | | |
| | | | | | ATCC | | | | | | |
| | | | | | 204508/ | | | | | | |
| | | | | | S288c) | | | | | | |
| | | | | | (Baker's | | | | | | |
| | | | | | yeast) | | | | | | |
| Cg24 | | 479015.32 | aspartate | P26 | Coryne- | | | | | | |
| A4A | | | kinase | 512 | bacterium | | | | | | |
| C_51 | | | activity | | glutamicum | | | | | | |
| Cg24 | | | diamino- | O52 | Halomonas | | | | | | |
| A4A | | | butyrate | 250 | elongata | | | | | | |
| C_52 | | | amino- | | (strain | | | | | | |
| | | | transferase | | ATCC | | | | | | |
| | | | | | 33173/ | | | | | | |
| | | | | | DSM | | | | | | |
| | | | | | 2581/ | | | | | | |
| | | | | | NBRC | | | | | | |
| | | | | | 15536/ | | | | | | |
| | | | | | NCIMB | | | | | | |
| | | | | | 2198/ | | | | | | |
| | | | | | 1H9) | | | | | | |
| Cg24 | | 354973.69 | diamino- | Q6 | Strepto- | | | | | | |
| A4A | | | butyrate | QU | myces | | | | | | |
| C_53 | | | amino- | Y9 | anulatus | | | | | | |
| | | | transferase | | (Strepto- | | | | | | |
| | | | | | myces | | | | | | |
| | | | | | chrysomallus) | | | | | | |
| Cg24 | | | Malate | Q12 | Saccharo- | | | | | | |
| A4A | | | dehydro- | 128 | myces | | | | | | |
| C_54 | | | genase | | cerevisiae | | | | | | |
| | | | | | (strain | | | | | | |
| | | | | | ATCC | | | | | | |
| | | | | | 204508/ | | | | | | |
| | | | | | S288c) | | | | | | |
| | | | | | (Baker's | | | | | | |
| | | | | | yeast) | | | | | | |
| Cg24 | | 502101.17 | aspartate | P26 | Coryne- | | | | | | |
| A4A | | | kinase | 512 | bacterium | | | | | | |
| C_55 | | | | | glutamicum | | | | | | |
| | | | | | (strain | | | | | | |
| | | | | | ATCC | | | | | | |
| | | | | | 13032/ | | | | | | |
| | | | | | DSM | | | | | | |
| | | | | | 20300/JCM | | | | | | |
| | | | | | 1318/LMG | | | | | | |
| | | | | | 3730/ | | | | | | |
| | | | | | NCIMB | | | | | | |
| | | | | | 10025) | | | | | | |
| Cg24 | | 406154.83 | aspartate | P23 | Saccharo- | | | | | | |
| A4A | | | amino- | 542 | myces | | | | | | |
| C_56 | | | transferase | | cerevisiae | | | | | | |
| | | | activity | | (strain | | | | | | |
| | | | | | ATCC | | | | | | |
| | | | | | 204508/ | | | | | | |
| | | | | | S288c) | | | | | | |
| | | | | | (Baker's | | | | | | |
| | | | | | yeast) | | | | | | |
| Cg24 | | 441737.02 | aspartate | N1 | Saccharo- | | | | | | |
| A4A | | | kinase | P4 | myces | | | | | | |
| C_57 | | | | U6 | cerevisiae | | | | | | |
| | | | | | (strain | | | | | | |
| | | | | | CEN.PK | | | | | | |
| | | | | | 113-7D) | | | | | | |
| | | | | | (Baker's | | | | | | |
| | | | | | yeast) | | | | | | |
| Cg24 | | 376681.66 | Aspartate- | P32 | Saccharo- | | | | | | |
| A4A | | | semi- | 801 | myces | | | | | | |
| C_58 | | | aldehyde | | cerevisiae | | | | | | |
| | | | dehydro- | | (strain | | | | | | |
| | | | genase | | ATCC | | | | | | |
| | | | (ASA | | 204508/ | | | | | | |
| | | | dehydro- | | S288c) | | | | | | |
| | | | genase) | | (Baker's | | | | | | |
| | | | (ASADH) | | yeast) | | | | | | |
| Cg24 | | | aspartate | P0 | Coryne- | | | | | | |
| A4A | | | semi- | C1 | bacterium | | | | | | |
| C_59 | | | aldehyde | D8 | glutamicum | | | | | | |
| | | | dehydro- | | (strain | | | | | | |
| | | | genase | | ATCC | | | | | | |
| | | | | | 13032/ | | | | | | |
| | | | | | DSM | | | | | | |
| | | | | | 20300/JCM | | | | | | |
| | | | | | 1318/LMG | | | | | | |
| | | | | | 3730/ | | | | | | |
| | | | | | NCIMB | | | | | | |
| | | | | | 10025) | | | | | | |
| Cg24 | | 437149.17 | Aspartate- | P0 | Coryne- | | | | | | |
| A4A | | | semi- | C1 | bacterium | | | | | | |
| C_60 | | | aldehyde | D8 | glutamicum | | | | | | |
| | | | dehydro- | | (strain | | | | | | |
| | | | genase | | ATCC | | | | | | |
| | | | (ASA | | 13032/ | | | | | | |
| | | | dehydro- | | DSM | | | | | | |
| | | | genase) | | 20300/JCM | | | | | | |
| | | | (ASADH) | | 1318/LMG | | | | | | |
| | | | | | 3730/ | | | | | | |
| | | | | | NCIMB | | | | | | |
| | | | | | 10025) | | | | | | |
| Cg24 | | 358002.03 | diamino- | A0A | Strepto- | | | | | | |
| A4A | | | butyrate | 087 | myces | | | | | | |
| C_61 | | | amino- | KB0 | sp. JS01 | | | | | | |
| | | | transferase | 8 | | | | | | | |
| Cg24 | | 649395.03 | diamino- | U4 | Glaesser- | | | | | | |
| A4A | | | butyrate | RV | ella | | | | | | |
| C_62 | | | amino- | R1 | parasuis | | | | | | |
| | | | transferase | | MN-H | | | | | | |
| Cg24 | | 604823.29 | diamino- | Q88 | Pseudo- | | | | | | |
| A4A | | | butyrate | F75 | monas | | | | | | |
| C_63 | | | amino- | | putida | | | | | | |
| | | | transferase | | (strain | | | | | | |
| | | | | | KT2440) | | | | | | |
| Cg24 | | 353252.09 | Aspartate- | P0 | Coryne- | Malate | Q8 | Coryne- | aspartate | P26 | Coryne- |
| A4A | | | semi- | C1 | bacterium | dehydro- | NN | bacterium | kinase | 512 | bacterium |
| C_64 | | | aldehyde | D8 | glutamicum | genase | 33 | glutamicum | activity | | glutamicum |
| | | | dehydro- | | (strain | | | (strain | | | |
| | | | genase | | ATCC | | | ATCC | | | |
| | | | (ASA | | 13032/ | | | 13032/ | | | |
| | | | dehydro- | | DSM | | | DSM | | | |
| | | | genase) | | 20300/JCM | | | 20300/JCM | | | |
| | | | (ASADH) | | 1318/LMG | | | 1318/LMG | | | |
| | | | | | 3730/ | | | 3730/ | | | |
| | | | | | NCIMB | | | NCIMB | | | |
| | | | | | 10025) | | | 10025) | | | |
| Cg24 | | 272981.73 | Aspartate- | P0 | Coryne- | Aspartate | Q8 | Coryne- | aspartate | P26 | Coryne- |
| A4A | | | semi- | C1 | bacterium | trans- | NT | bacterium | kinase | 512 | bacterium |
| C_65 | | | aldehyde | D8 | glutamicum | aminase | R2 | glutamicum | activity | | glutamicum |
| | | | dehydro- | | (strain | | | (strain | | | |
| | | | genase | | ATCC | | | ATCC | | | |
| | | | (ASA | | 13032/ | | | 13032/ | | | |
| | | | dehydro- | | DSM | | | DSM | | | |
| | | | genase) | | 20300/JCM | | | 20300/JCM | | | |
| | | | (ASADH) | | 1318/LMG | | | 1318/LMG | | | |
| | | | | | 3730/ | | | 3730/ | | | |
| | | | | | NCIMB | | | NCIMB | | | |
| | | | | | 10025) | | | 10025) | | | |
| Cg24 | | 347820.1 | Malate | Q8 | Coryne- | | | | | | |
| A4A | | | dehydro- | NN | bacterium | | | | | | |
| C_66 | | | genase | 33 | glutamicum | | | | | | |
| | | | | | (strain | | | | | | |
| | | | | | ATCC | | | | | | |
| | | | | | 13032/ | | | | | | |
| | | | | | DSM | | | | | | |
| | | | | | 20300/JCM | | | | | | |
| | | | | | 1318/LMG | | | | | | |
| | | | | | 3730/ | | | | | | |
| | | | | | NCIMB | | | | | | |
| | | | | | 10025) | | | | | | |
| Cg24 | | | diamino- | A0A | Photo- | | | | | | |
| A4A | | | butyrate | 090 | bacterium | | | | | | |
| C_67 | | | amino- | R3 | aphoticum | | | | | | |
| | | | transferase | G5 | | | | | | | |

TABLE 2
Engineering of Corynebacterium glutamicum to Produce Ectoine
This table shows the results of engineering one of the best-performing strains from Example 3, which was
Cg2A4AC_63 (shown in the top row), containing one copy of a gene encoding diaminobutyrate aminotransferase
from Glaesserella parasuis MN-H (Uniprot ID U4RVR1 and one copy of a gene encoding diaminobutyrate
aminotransferase from Pseudomonas putida (Uniprot ID Q88F75). “Enzyme 1,” “E1,” “Enzyme 2,” and “E2”
below refer to enzymes expressed in addition to these two enzymes.

| | | E1 | Enzyme 1- | Enzyme 1- | E2 | Enzyme 2- | Enzyme 2- |
|---|---|---|---|---|---|---|---|
| Strn. | | Uniprot | activity | source | Uniprot | activity | source |
| name | Titer (μg/L) | ID | name | organism | ID | name | organism |
| Cg24 | 236.661775 | | | | | | |
| A4A | | | | | | | |
| C_63 | | | | | | | |
| CgE | 7483.799525 | Q2SLV8 | L-ectoine | Hahella | A0A090I5X2 | L-2,4- | Methano- |
| CTOI | | | synthase | chejuensis | | diamino- | bacterium |
| N_01 | | | | (strain | | butyric | formicicum |
| | | | | KCTC | | acid acetyl- | |
| | | | | 2396) | | transferase | |
| | | | | | | EctA | |
| CgE | 26404.85238 | A0A0Q1AWK1 | Uncharacter- | Methanosaeta | B4R981 | L-ectoine | Phenylo- |
| CTOI | | | ized | sp. SDB | | synthase | bacterium |
| N_02 | | | protein | | | | zucineum |
| | | | | | | | (strain HLK1) |
| CgE | 115805.2853 | Q9KED5 | L-ectoine | Bacillus | F4C028 | L-2,4- | Methanosaeta |
| CTOI | | | synthase | halodurans | | diamino- | concilii (strain |
| N_03 | | | | (strain ATCC | | butyric | ATCC 5969/ |
| | | | | BAA-125/ | | acid acetyl- | DSM 3671/ |
| | | | | DSM 18197/ | | transferase | JCM 10134/ |
| | | | | FERM 7344/ | | | NBRC 103675/ |
| | | | | JCM 9153/ | | | OCM 69/ |
| | | | | C-125) | | | GP-6) |
| | | | | | | | (Methanothrix |
| | | | | | | | concilii) |
| CgE | 31689.86435 | Q1GNW6 | L-ectoine | Sphingopyxis | A9A2K3 | L-2,4- | Nitrosopumilus |
| CTOI | | | synthase | alaskensis | | diamino- | maritimus |
| N_04 | | | | (strain DSM | | butyric | (strain SCM1) |
| | | | | 13593/ | | acid acetyl- | |
| | | | | LMG 18877/ | | transferase | |
| | | | | RB2256) | | | |
| | | | | (Sphingomonas | | | |
| | | | | alaskensis) | | | |
| CgE | 504683.1859 | E6W3G0 | L-2,4- | Desulfurispirillum | Q0A9E8 | L-ectoine | Alkalilimnicola |
| CTOI | | | diamino- | indicum | | synthase | ehrlichii |
| N_05 | | | butyric | (strain ATCC | | | (strain ATCC |
| | | | acid acetyl- | BAA-1389/S5) | | | BAA-1101/ |
| | | | transferase | | | | DSM 17681/ |
| | | | | | | | MLHE-1) |
| CgE | 0 | W4LG62 | Uncharacter- | Candidatus | F8E4K4 | L-2,4- | Flexistipes |
| CTOI | | | ized | Entotheonella | | diamino- | sinusarabici |
| N_06 | | | protein | sp. TSY1 | | butyric | (strain |
| | | | | | | acid acetyl- | DSM 4947/ |
| | | | | | | transferase | MAS 10) |
| CgE | 86929.57735 | W9UWT5 | L-ectoine | Nitrincola | A0A059XSI6 | L-2,4- | Leptospirillum |
| CTOI | | | synthase | nitratireducens | | diamino- | ferriphilum |
| N_07 | | | | | | butyric | YSK |
| | | | | | | acid acetyl- | |
| | | | | | | transferase | |
| CgE | 15770.48973 | A0A1N6PYR6 | L-ectoine | Marinobacterium | A0A0J6YS11 | Uncharacter- | Nitrospina |
| CTOI | | | synthase | stanieri | | ized | sp. SCGC_ |
| N_08 | | | | | | protein | AAA799_ |
| | | | | | | | A02 |
| CgE | 125987.246 | A0A1Q9TDA5 | L-ectoine | Actinomadura | H9UMW7 | Diamino- | Spirochaeta |
| CTOI | | | synthase | sp. CNU-125 | | butyrate | africana |
| N_09 | | | | | | acetyl- | (strain |
| | | | | | | transferase | ATCC |
| | | | | | | | 700263/ |
| | | | | | | | DSM 8902/ |
| | | | | | | | Z-7692) |
| CgE | 55686.12093 | A0A1G2X194 | L-ectoine | Phenylobacterium | H3SPK2 | L-2,4- | Paenibacillus |
| CTOI | | | synthase | sp. | | diamino- | dendritiformis |
| N_17 | | | | RIFCSPHIGHO2_ | | butyric | C454 |
| | | | | 01_FULL_ | | acid acetyl- | |
| | | | | 70_10 | | transferase | |
| CgE | 78449.77135 | D8K1W8 | L-2,4- | Dehalogenimonas | A0A059XT79 | L-ectoine | Lepto- |
| CTOI | | | diamino- | lykanthro- | | synthase | spirillum |
| N_10 | | | butyric | porepellens | | | ferriphilum |
| | | | acid acetyl- | (strain ATCC | | | YSK |
| | | | transferase | BAA-1523/ | | | |
| | | | | JCM 15061/ | | | |
| | | | | BL-DC-9) | | | |
| CgE | 11060.46955 | A0A0S8C710 | L-ectoine | Betaproteobacteria | Q7WHI7 | L-2,4- | Bordetella |
| CTOI | | | synthase | bacterium | | diamino- | bronchiseptica |
| N_11 | | | | SG8_41 | | butyric | (strain ATCC |
| | | | | | | acid acetyl- | BAA-588/ |
| | | | | | | transferase | NCTC |
| | | | | | | | 13252/RB50) |
| | | | | | | | (Alcaligenes |
| | | | | | | | bronchisepticus) |
| CgE | 551070.1294 | A0A160TCS0 | L-ectoine | hydrothermal | Q4JQJ5 | L-2,4- | Methylo- |
| CTOI | | | synthase | vent | | diamino- | microbium |
| N_13 | | | | metagenome | | butyric | alcaliphilum |
| | | | | | | acid acetyl- | (strain DSM |
| | | | | | | transferase | 19304/ |
| | | | | | | | NCIMB |
| | | | | | | | 14124/ |
| | | | | | | | VKM B-2133/ |
| | | | | | | | 20Z) |
| CgE | 60632.42763 | A0A1V2SL65 | L-ectoine | Bacillus | Q9KED3 | L-2,4- | Bacillus |
| CTOI | | | synthase | sp. VT-16-64 | | diamino- | halodurans |
| N_14 | | | | | | butyric | (strain ATCC |
| | | | | | | acid acetyl- | BAA-125/ |
| | | | | | | transferase | DSM 18197/ |
| | | | | | | | FERM 7344/ |
| | | | | | | | JCM 9153/ |
| | | | | | | | C-125) |
| CgE | 355969.1187 | A0A081BBU4 | L-ectoine | Tepidicaulis | Q6QUZ0 | L-2,4- | Streptomyces |
| CTOI | | | synthase | marinus | | diamino- | anulatus |
| N_15 | | | | | | butyric | (Streptomyces |
| | | | | | | acid acetyl- | chrysomallus) |
| | | | | | | transferase | |
| CgE | 120148.4699 | Q8ESU9 | L-2,4- | Oceanobacillus | J9Z7Y9 | L-ectoine | Leptospirillum |
| CTOI | | | diamino- | iheyensis | | synthase | ferriphilum |
| N_16 | | | butyric | (strain | | | (strain ML-04) |
| | | | acid acetyl- | DSM 14371/ | | | |
| | | | transferase | CIP 107618/ | | | |
| | | | | JCM 11309/ | | | |
| | | | | KCTC 3954/ | | | |
| | | | | HTE831) | | | |

Note:
The enzyme activity name “uncharacterized protein refers to the fact that some enzymes tested were poorly annotated in a public database. These enzyme were selected as described in WO2020037085A1. That strains expressing these enzymes make ectoine indicates that they provide an enzyme activity required for producing ectoine (e.g., for strain CgECTOIN_02, the enzyme having Uniprot ID A0A0Q1AWK1 functions as an L-ectoine synthase.

What is claimed is:

1. An engineered microbial cell that produces diaminobutyric acid, wherein the engineered microbial cell expresses a non-native diaminobutyrate aminotransferase.

2. The engineered microbial cell of claim 1, wherein the engineered microbial cell expresses a non-native L-2,4-diaminobutyrate acetyltransferase and produces N-acetyl-2,4-diaminobutyrate.

3. The engineered microbial cell of claim 2, wherein the engineered microbial cell expresses a non-native ectoine synthase and produces ectoine.

4. The engineered microbial cell of any one of claims 1-3, that comprises increased activity of at least one or more upstream pathway enzyme(s) leading to L-aspartate-4-semialdehyde, said increased activity being increased relative to a control cell.

5. The engineered microbial cell of claim 4, wherein the one or more upstream pathway enzyme(s) are selected from the group consisting of aspartate semi-aldehyde dehydrogenase, apartokinase, aspartate aminotransferase, diaminobutyrate aminotransferase, and malate dehydrogenase.

6. The engineered microbial cell of any one of claims 1-5, wherein the engineered microbial cell is a bacterial cell.

7. The engineered microbial cell of claim 6, wherein the bacterial cell is a cell of the genus Corynebacterium.

8. The engineered microbial cell of claim 7, wherein the bacterial cell is a cell of the species glutamicum.

9. The engineered microbial cell of claim 8, wherein the non-native diaminobutyrate aminotransferase has at least 70% amino acid sequence identity with:
a Glaesserella parasuis MN-H diaminobutyrate aminotransferase comprising SEQ ID NO:1; and/or
a Pseudomonas putida diaminobutyrate aminotransferase comprising SEQ ID NO:2.

10. The engineered microbial cell of claim 9, wherein:
the Glaesserella parasuis MN-H diaminobutyrate aminotransferase comprises SEQ ID NO:1; and/or
the Pseudomonas putida diaminobutyrate aminotransferase comprises SEQ ID NO:2.

11. The engineered microbial cell of any one of claims 1-10, wherein, when cultured, the engineered microbial cell produces diaminobutyric acid at a level of at least 500 mg/L of culture medium.

12. The engineered microbial cell of any one of claims 9-11, wherein the engineered microbial cell expresses a non-native L-2,4-diaminobutyrate acetyltransferase and produces N-acetyl-2,4-diaminobutyrate.

13. The engineered microbial cell of claim 12 wherein the non-native a L-2,4-diaminobutyrate acetyltransferase has at least 70% amino acid sequence identity with:
a Methylomicrobium alcaliphilum (strain DSM 19304/NCIMB 14124/VKM B-2133/20Z) L-2,4-diaminobutyrate acetyltransferase comprising SEQ ID NO:3; and/or
a Desulfurispirillum indicum (strain ATCC BAA-1389/S5) L-2,4-diaminobutyrate acetyltransferase comprising SEQ ID NO:4.

14. The engineered microbial cell of claim 13, wherein:
the Methylomicrobium alcaliphilum (strain DSM 19304/NCIMB 14124/VKM B-2133/20Z) L-2,4-diaminobutyrate acetyltransferase comprises SEQ ID NO:3; and/or
the Desulfurispirillum indicum (strain ATCC BAA-1389/S5) L-2,4-diaminobutyrate acetyltransferase comprises SEQ ID NO:4.

15. The engineered microbial cell of claim 13 or claim 14, wherein the engineered microbial cell expresses a non-native ectoine synthase and produces ectoine.

16. The engineered microbial cell of claim 15, wherein the non-native ectione synthase has at least 70% amino acid sequence identity with:
a hydrothermal vent metagenome ectione synthase comprising SEQ ID NO:5; and/or
an Alkalilimnicola ehrlichii (strain ATCC BAA-1101/DSM 17681/MLHE-1) ectione synthase comprising SEQ ID NO:6.

17. The engineered microbial cell of claim 16, wherein:
the hydrothermal vent metagenome ectione synthase comprises SEQ ID NO:5; and/or
the Alkalilimnicola ehrlichii (strain ATCC BAA-1101/DSM 17681/MLHE-1) ectione synthase comprises SEQ ID NO:6.

18. The engineered microbial cell of claim 17, wherein:
the hydrothermal vent metagenome ectione synthase comprises SEQ ID NO:5, and the non-native L-2,4-diaminobutyrate acetyltransferase comprises a Methylomicrobium alcaliphilum (strain DSM 19304/NCIMB 14124/VKM B-2133/20Z) L-2,4-diaminobutyrate acetyltransferase comprising SEQ ID NO:3; and/or
the Alkalilimnicola ehrlichii (strain ATCC BAA-1101/DSM 17681/MLHE-1) ectione synthase comprises SEQ ID NO:6, and the non-native L-2,4-diaminobutyrate acetyltransferase comprises a Desulfurispirillum indicum (strain ATCC BAA-1389/S5) L-2,4-diaminobutyrate acetyltransferase comprising SEQ ID NO:4.

19. The engineered microbial cell of claim 18, wherein, when cultured, the engineered microbial cell produces ectoine at a level of at least 500 mg/L of culture medium.

20. A method of culturing engineered microbial cells according to any one of claims 1-19, the method comprising culturing the cells under conditions suitable for producing diaminobutyric acid, optionally wherein:
the engineered microbial cells produce diaminobutyric acid at a level of at least 500 mg/L of culture medium;
the engineered microbial cells produce N-acetyl-2,4-diaminobutyrate at a level of at least 500 mg/L of culture medium;
the engineered microbial cells produce ectoine at a level of at least 500 mg/L of culture medium; and/or
the method additionally comprises recovering diaminobutyric acid, N-acetyl-2,4-diaminobutyrate, or ectoine, respectively, from the culture.


SEQUENCE LISTING (101 sequences)

SEQ ID NO: 1 - protein, 454 aa, Unknown
  Description of Unknown Glaesserella parasuis sequence
MTTIPVNPINQATNEYYLTRQNEMESNVRSYPRKLPLAIAKAQGCWVTDVEGNEYLDCLAGAGTLALGHNHPAVIQAIQDVLASGLPLHTLDLTTPLKDAFTEELLSFFPKGEYRLQFCGPSGADGTEAAIKLAKTYTGRGNVIAFSGGYHGMTHGSLSMTGNLNAKNAVQNLMAGVQFMPYPHEYRCPLGIGGEAGVDALTYYFENFIEDVESGVVKPAAVILEAIQGEGGVVTAPVKWLQKIREVTKKHGIVLILDEVQAGFCRSGKMFAFEHAGIEPDVVVMSKAVGGSLPLAVLAIKKEFDAWQPAGHTGTFRGNQLAMATGYVSLKIMREQNLAKNAEERGAFLKAELQKLAQQYPCIGNVRGKGLMMGFEIVDERQPQDRIGSYPADGGLAAEIQKACFKNKLLLERGGRCGNVVRILCPLIITQAECEEMIKRFSKSVADALNTVRG

SEQ ID NO: 2 - protein, 452 aa, Pseudomonas putida
MPQPLYEFTDSPLLQRQQQQESNARSYPRRIPLALRRARGIHVEDVEGRQFIDCLAGAGTLALGHNHPVVVEAIQRVLADELPLHTLDLTTPVKDRFVQDLFGILPEALRREAKVQFCGPTGTDAVEAALKLVRTATGRSTVLAFQGAYHGMSQGALNLMGSHGPKQPLGALLGNGVQFMPYPYDYRCPFGLGGEAGVKANLHYLENLLLDPESGVPLPAAVILEVVQGEGGVVPADIEWLKGVRRITEQAGVALIVDEIQSGFARTGRMFAFEHAGIVPDVVTLSKAIGGSLPLAVVVYRDWLDTWKPGAHAGTFRGNQMAMAAGSAVINYLVEHRLAEHAEAMGQRLRGHLQRLQRDYPQLGDIRGRGLMLGVELVDPQGQPDALGHPPANRDLAPKVQRECLKRGLILELGGRHGAVVRFLPPLIISAEQIDEVAQRFSAAVAAAVGSV

SEQ ID NO: 3 - protein, 172 aa, Methylomicrobium alcaliphilum
MLPDKTALPIITLSQPTAEVGAQVHRLISKCPPLDPNSMYCNLLQSSHFSETAVAAKIGDELVGFVSGYRIPQRPDTLFVWQVAVGEKARGQGLATRMLKAILARPVNQDINRIETTITPNNKASWALFEGLAKKLDTQIGSAVMFDKTRHFADQHETEMLVKVGPFKAVQA

SEQ ID NO: 4 - protein, 183 aa, Desulfurispirillum indicum
MIADKKHFERFTMRQTGDTDPSTITLTTPTLADGAEVYRLISQCPPLDVNSQYCYHIICEHFSQTSVIARIDGDAAGFISGYTLPEHPDILFIWQVAVSAKARGRQLATRMLEHIICRKRATAVRFLHTTISPSNIPSQKLFSAIARTFKAPMREREFLAGEVFAEGHEAEVLYEIGPLQYSQ

SEQ ID NO: 5 - protein, 132 aa, Unknown
  Description of Unknown hydrothermal vent metagenome sequence
MIVRTLKDCKNSERAINGGTWESVRMLLKNDNMGFSFHITTIFANTETPIWYQNHLESVYCMSGRGEVETVADGKIYPIEPGTLYILDKNDKHLLRASEDMEMACVFNPPLNGKEVHDENGVYALDGETLTD

SEQ ID NO: 6 - protein, 132 aa, Alkalilimnicola ehrlichii
MKIVKLQDIIGTEREVHGEGWRSRRILLKKDKMGFSLHETIWPPGHELRMWYKNHLEAVYCVAGNGSIEDLETGEVHELYDGVCYALDKHDRHILRGGTEEMRLVCVFNPPVTGLEVHDEDGAYCLVEDDDD

SEQ ID NO: 7 - protein, 170 aa, Methanobacterium formicicum
MGPENDENQPVKIRTPQIKDGNQIYHLVKKSKPLDINSLYSYLLICTHFDETSAVAEWNNKIIGFISAYINPHQENNLFIWQVAVLPTMRGQGLATQMIEHILHRKETQSIEFIETTVTPTNDASMALFKNIAANLDTNLEKTSFFTRELFGNPHHEEELLLQIGPLNNK

SEQ ID NO: 8 - protein, 132 aa, Hahella chejuensis
MIVRTLEQARQSDRRVKADNWESVRMLLKDDNMGFSFHITTLYANKETPIHYQNHLESVYCISGEGEVETVADGEVHAIKPGTLYVLDKHDKHLLRAFSEMTVACVFNPPLNGKETHDENGVYPLEAETIME

SEQ ID NO: 9 - protein, 171 aa, Methanosaeta sp.
MRASLRRTKEFVLRKPEVSDGAKIFNLVEICKPLDLNSVYSYLLLCHHFADTCVVAELDGEIFAFLSGYRPPDQEGVFFVWQVAVDPQMRGQGLGKLLLTETLKRPAVRGCNFLETTITPSNNASQRLFTSLARDLKARCTVSSCFSDEHFGQENHEAEHLFRIGPFSLSG

SEQ ID NO: 10 - protein, 130 aa, Unknown
  Description of Unknown Phenylobacterium zucineum sequence
MIVRDLSQAEKTDRRVVSKGWESTRLLLKDDAMGFSFHITTIYEGAELPMHYKNHLEAVYCISGEGSIEDLATGQVHEIRPGVLYALDKHDRHVLRGRTQMVMACAFNPPVTGREVHDETGAYPLEAEMA

SEQ ID NO: 11 - protein, 176 aa, Methanothrix soehngenii
MKSDQGSSDSEDVGRLKFRKPRLNDGARISELIRSCKPLDINSTYCYLLLCRDFADTCVVAEEGGEIVAFLSGYRPPDRWDVFFIWQIAVDVDSRNRGIAKRMLFDVLNRPSTRDCRYIETTISPSNEASKRLFRGLAQELDAHCDVSDCFSESDFGDFDHEAECLFNIGPFKMNG

SEQ ID NO: 12 - protein, 129 aa, Bacillus halodurans
MKVVKLEDVIGTEQEVKGENWTSRRLLLKKDGMGYSVHDTIIKAGTETHIWYQNHLEAVYCIEGEGEVETVKDGKVWPIKANEIYALDEHDEHLLRAKTDMRMVCVFNPPITGKETHDENGVYPLVDDE

SEQ ID NO: 13 - protein, 167 aa, Unknown
  Description of Unknown Nitrosopumilus maritimus sequence
MTSITFREPRVDDANKIWELVQNNKPLDENSKYLYVLLCHQFSKTCVVAEYDSKIVGFLSGFISPKNHDTLFVWQAAVDDSYRNKGVAKELVFKAFSQTDYDVRFVEATVTPSNKASLKFLQNFAAQLDADFVKTPLFSSDVLGDGHEQEDLVRIGPIQKSLLEVTA

SEQ ID NO: 14 - protein, 137 aa, Sphingopyxis alaskensis
MIVRNLGDIRKTDRNVRSDGWASARMLLKDDGMGFSFHVTTLFAGSELRMHYQNHLEAVLVLKGTGTIEDLATGEVHALRPGVMYALDDHDRHIVRPETDILTACVFNPPVTGREVHDESGAYPADPELAREPVAAD

SEQ ID NO: 15 - protein, 277 aa, Unknown
  Description of Unknown Candidatus Entotheonella sp. sequence
MLIRTMDQLEAEGRVISISHGRATAVRLLTKSDGLGFSVSEARATQAGQSDLWYKHHWEANYVRSGRATLEDRSTGERWQLEPGVLYCVGPRDRHRIIRDEPSDMRIISVFNPPIEGDETHDADGAYPPTGEPPRGQDRMFVKTLADVRQAGMERTVANGKAVSARYLTVDDHLGFSLNSVHLKAGSEADLWYKHHWEANLVLDGTLDVIDYATGDVHRLGPGGLYLVGPKDRHHVKVLSDFHGLSVFNPPLTGTEDHDADGSYPPTGPVPPGPAGG

SEQ ID NO: 16 - protein, 167 aa, Flexistipes sinusarabici
MEDNIIIKRPVVKDAFGIWTLIKNSEPLDLNSKYAYMLFCTHFRDTSVVAIDPDQDDKVVGFVSGYRPPVQPDALFVWQVVVDSSMRGRGLAPRIIIDILEREYNLDINYIQATVGPSNKPSEGLFRKLAKYYDAKCETSVFFSKEDFGEGDHEEEVLFHIGPIKRG

SEQ ID NO: 17 - protein, 187 aa, Leptospirillum ferriphilum
MNSENISPVLSDFQAPSRRETERLFRFRTPTDLDGQSVHRLIARCPPLDVNSLYANLLQCLHFSGTSILAETGDGQLAAFISGYVIPENPDTLFIWQVAVDPVHRGKGVALSMLEALLDRTLSMGVRYLETTISPENGPSRKLFQKLFSRRKAPFSRRPLFGRETHFGGGHDDEVLYRGGPFSAVGP

SEQ ID NO: 18 - protein, 129 aa, Unknown
  Description of Unknown Nitrincola nitratireducens sequence
MIVRHLKDCEASERRVVSENWESTRMLLKDDQMGFSFHITTIYANTETHIWYQNHLESVYCMSGTGEIETLADGKVYPIEPGMLYILDQHDEHLLRATTEMKMACVFNPPLSGKEVHDENGVYPVDDSK

SEQ ID NO: 19 - protein, 160 aa, Nitrospina sp.
MQLRKPSLEDGPRMFDLARSAGNLDVNSCYHYLLLSQKFAETCAVAVQGEKLMGFATGFRDPNKPEILFIWQVAVSDDFRNGGVAKAMLNHILQRNFSPPIQFLEVTIAPSNKASQALFQSLANELNCSIENESLISADLFPENGHEPESLFRIGPLNCH

SEQ ID NO: 20 - protein, 132 aa, Marinobacterium stanieri
MIVRTLEECENSERRIVSKTWESTRMLLKDDNMGYSFHITTIYAGTETHIWYQNHLESVYCISGEGEIETLADGKIYPIKPGTLYNLNEHDEHLLRADKELKLACVFNPPLNGKEVHDENGVYPLEADTVEA

SEQ ID NO: 21 - protein, 174 aa, Spirochaeta africana
MNFRHPTAEDGAAIWRIVRDSGVLDLNSSYCYLMMGHYFGDTCIVAEDPHTDPAANGDRLAGFIVGFRPPQDPATLFVWQVAVDPGHRGKGVGSRMLQALLAAQPAEEIHYIEATISPSNTASQALFRKAAATLKTDCSVSDLFLADMFPGSGEHEPEHRYRIGPFTLSNSANQ

SEQ ID NO: 22 - protein, 131 aa, Actinomadura sp.
MIVRSLKEIIGTERDVQAPTWHSRRFVLAKDGVGFSMHETVLYAGTETKMWYANHIEAVYCIEGQGELINHENGETHRIEPGVMYLLNEHDHHTLRAHTDVKTVCVFNPPVTGREVHDENGVYPLLTEEDA

SEQ ID NO: 23 - protein, 167 aa, Dehalogenimonas lykanthroporepellens
MKINYRQPDIKDGLDIYRLVQSCPPLDLNSAYCYLLLATHFADTCVIAEDTTAGKIAGFVSAYFRPDAPRTLFVWQIAVHPDYRGQGIARGLTTALVEAAYRQTTPPEYLELSVNPSNQASRGLFTRLAEQLGAELTESVLFSEELLGGGHEAEIMLRIGPLKGDPQ

SEQ ID NO: 24 - protein, 131 aa, Leptospirillum ferriphilum
MIVRTLEEAERSERRVVTPTWESTRLLLKNDRMGFSFHITTIYANTETHICYQNHLEAVYCISGEGEVETLADGVVHKIRPGTIYVLDRHDDHLLRGITEMKLACVFNPPLSGKEVHDEEGVYPLEAEPIS

SEQ ID NO: 25 - protein, 186 aa, Bordetella bronchiseptica
MRKDETSNTSPDISAAQPASALRYQLRPPRRNDGAAIHQLVSECPPLDLNSLYAYLLLCEHHAHTCVVAESPGGRIDGFVSAYLLPTRPDVLFVWQVAVHSRARGHRLGRAMLGHILERQECRHVRHLETTVGPDNQASRRTFAGLAGERGAHVSEQPFFDRQAFGGADHDDEMLLRIGPFTHPPH

SEQ ID NO: 26 - protein, 136 aa, Unknown
  Description of Unknown Betaproteobacteria bacterium SG8_41 sequence
MILRSLDEVKGTSRDVETPNWVSRRLLLRSDGMGFSLHETTVYPGTETYIWYANHLEAVYCVEGEGEVETIPDKKIYPIRPGVVYALDQNDKHYLRARTKMRLVCVFNPPLTGREVHDENGVYPLVTDEAAAARAS

SEQ ID NO: 27 - protein, 275 aa, Unknown
  Description of Unknown hydrothermal vent metagenome sequence
MFIRSLRDVKDTDRYKVLPKSATHTARYLTAADGMGFSLHVNRGKAIPPTQLWYKNHWEANYLIAGVIKVTDMTSGQEWLLESGDLYQVGPNDRHLYEVLEDEHHLSIFCPSLWGDETHDEDGAYSESGPVPVTEQRMFVRHIEHMREAGREMLVANGQARVVRALTKTDGLGFSFSDVNFAAGATTKLWYKHHWETNYIISGTGQVKDLTTGESWKLEPDMTYNVGPKDQHQLSAETNLHLASVFCPPLEGTEQHDSDGALEPSGPVPPGPPGY

SEQ ID NO: 28 - protein, 166 aa, Unknown
  Description of Unknown Deltaproteobacteria bacterium ADurb.BinA014 sequence
MKNDSHHILFRLPEKKDAQAVRRLIEDSPPLDLNSLYCYLLLCTHFAPTSAIAEYHQEVCGFVSAYIRPDQKDTLFVWQVAVQRDFRGKSIAKGMLLSCLQRAHLSSLRYLETTVNPSNTSSNALFSSLAAFLNAGLTKSVLFSADDFGKMSHEEEILYRIGPFNI

SEQ ID NO: 29 - protein, 189 aa, Bacillus halodurans
MQVQCNEKAFKGGFIINSQIATAPPKTLDTTITIGKPTVEDGAAMWELVNKSTLDTNSPYKYIMMCEYFAETCVVAKENERLVGFVTAFIPPEHQDVIFVWQIGVDSSQRGKGLASKLLQELISRDICSNVNYVEATVTPSNKASQALFQKLAREYNTQCEVSECFSEDLFPGDDHEAELTFRIGPLHP

SEQ ID NO: 30 - protein, 128 aa, Bacillus sp.
MIVRTLDEIIGTENEVKADTWASRRLLLKSDNMGFSFHDTIIYAGTETHIWYKNHLEAVYCVGGEGEIETVSDGKVYPLKDGTMYALDQNDEHYLRATKDMRMICVFNPPLTGRETHDKDGVYPLVED

SEQ ID NO: 31 - protein, 176 aa, Streptomyces anulatus
MTAAPADFARARSEFLSIDAPRVEDGAAIWRIARDSQVLDLNSSYSYLLWCRDFAATSAVARGENGEPIAFVTGYVRPDRPQTLVVWQVAVDQAHRGKGLAAALLDALTARVAADQVLSSVETTITPDNTASDRLFTSYAQRHDVALEKEVLFDGELFPEETHLPEVLYRIGPFAT

SEQ ID NO: 32 - protein, 128 aa, Unknown
  Description of Unknown Tepidicaulis marinus sequence
MIVRTLEEAEKSDRKVQSKTWDSVRLLLKDDNMGFSFHITTLYAGTETRMHYKNHLESVYCMEGEAEIETLADGKKYEIKPGTIYILDQHDEHILRVHKEVKMACVFNPPVTGQEVHDESGAYPLVVD

SEQ ID NO: 33 - protein, 179 aa, Oceanobacillus iheyensis
MAEKLKVDTDEYYFRRPTKEDGGPVWELVREIGNLDLNSSYSYVLWCEVFAESSIVVEHKETQQIVGFISGFMHPEKEDTLFIWQVAVSSTQRGKGLATKMLLQLLEWNESVNFIEATVAPSNKPSNYLFLGLARKIHTNWKISDYFKTDHFPAKDEEHEEELLFRIGPMRKNKNKRMI

SEQ ID NO: 34 - protein, 131 aa, Leptospirillum ferriphilum
MIVRTLEEAERSERRVVTPTWESTRLLLKNDRMGFSFHITTIYANTETHICYQNHLEAVYCISGEGEVETLADGVVHKIRPGTLYVLDRHDDHLLRGITEMKLACVFNPPLSGKEVHDEKGVYPLEAEPIS

SEQ ID NO: 35 - protein, 179 aa, Paenibacillus dendritiformis
MNVNVTPSLPCTIRNARKGDGTELWKLVKESGTLDVNSAYCYIMLCDYFADTCIVAEHNRNIIGFVSALRRPDMPETLFVWQIAVRDRYRGRGIAEALLRTLYDTACRDQVRFIETTITPSNTASQRLFAKMAKTWKSALVTKVGFASQLFPDGAHETEKLIRIGPLRHNTHDRMETVT

SEQ ID NO: 36 - protein, 130 aa, Phenylobacterium sp.
MIVRDLNEANQTRRRVVSKGWESTRLLLKDDQMGFSFHITTIYAGAELPMHYQNHLESVYCISGEGSIEDTATGEVHEIKPGVMYALDKHDKHILRGKTEMKMACVFNPPLNGLEVHDDTGAYPLEAETV

SEQ ID NO: 37 - DNA, 1362 bp, Unknown
  Description of Unknown Glaesserella parasuis sequence
atgactacgattcccgtgaacccaattaatcaggccacaaacgaatactatttgacccgacagaacgagatggagtcgaatgttcgttcctatcctcgcaaactcccactggctatcgccaaggcccaaggctgttgggttacagacgtagaaggcaacgagtacctcgattgcttggccggcgctggtacactcgcgttgggccataaccatccggccgttattcaggccatccaagatgtgttggcctccggcttgcccctgcacaccctggatctcacgacccccctcaaagacgcattcacagaagagctcctctccttcttcccaaagggagaataccgtctccagttctgcggaccgtcaggcgccgatggtactgaggcggcaattaagttggctaaaacctacaccggccgcggcaacgtgattgcgttctcaggcggataccatggtatgacccatggctccctctccatgacgggtaacctcaatgcaaagaacgccgtgcagaacctgatggcaggagtgcagtttatgccgtatccgcatgagtaccgctgtccactcggaattggaggcgaagcaggcgtcgatgcattgacctactacttcgagaactttattgaagatgttgaatcgggtgttgtgaaaccagccgctgtgatcctggaagctattcagggcgaaggcggtgttgtgaccgctcccgtgaagtggctccaaaagattcgcgaggttacaaagaagcacggcattgtcctgattcttgatgaggtgcaggctggcttctgccgttcaggcaagatgttcgcctttgaacacgcaggaatcgaaccagacgtcgtcgtcatgtccaaggcggttggtggctccctccccctggctgtactggcgatcaagaaggagtttgatgcttggcagccagccggccacactggcactttccgcggaaaccagctggcgatggcaaccggttacgtctcacttaagattatgcgcgaacagaaccttgctaagaatgccgaggagcgtggcgctttcctgaaggcagagctccaaaagctcgcgcaacagtatccatgcatcggaaacgtccgcggtaagggcctgatgatgggatttgaaatcgtcgacgaacgtcagcctcaagatcgcattggcagctaccctgctgatggcggcctggccgccgagatccagaaggcttgtttcaagaataagctgctgcttgagcgcggtggtcgctgtggtaacgttgtgcgcatcctctgccctctcatcatcacgcaggctgaatgcgaggaaatgatcaaacgcttttccaagtcagtggccgacgctcttaacaccgtccgtggc

SEQ ID NO: 38 - DNA, 1362 bp, Unknown
  Description of Unknown Glaesserella parasuis sequence
atgaccacgattccagtgaacccaatcaatcaagcgactaatgaatactacttgactcgtcaaaacgagatggagagcaacgtgcgctcctacccccgcaaactgcctctcgccattgccaaggcgcagggttgctgggtcactgacgttgagggcaacgaatacctggattgtctggctggcgctggtacgcttgccctgggtcacaatcatcccgcggtcattcaagccattcaggatgtcctcgcgagcggtcttccccttcataccctggacctcaccaccccactgaaggacgcatttacggaggaacttttgtccttctttcctaagggtgaataccgcctgcagttttgtggcccgtccggtgccgatggtaccgaagctgccattaaactcgcaaagacctataccggccgcggcaacgtcatcgcatttagcggcggctaccacggaatgacccacggctccctgtccatgacgggcaaccttaatgccaagaacgctgtgcagaacctcatggcaggagtgcagttcatgccgtatcctcacgaataccgctgcccacttggaatcggtggagaagctggcgtggacgccctcacctattacttcgagaacttcattgaagacgtcgagtcaggagtagttaaaccggccgccgttatcctggaagctatccaaggcgaaggtggcgtcgtgactgctccagtgaaatggctccagaaaattcgtgaagtcaccaagaaacacggcatcgtcttgatcctcgacgaagtgcaggctggattctgccgatcaggtaagatgtttgcattcgaacacgcgggaatcgaaccagatgttgtggttatgtcgaaggctgtaggcggttccctcccgttggcggtgttggcgattaaaaaggagttcgatgcatggcagcctgcgggtcacaccggcacttttcgtggcaaccagttggccatggcaaccggctacgtgtccctgaaaattatgcgtgaacagaacctggctaaaaatgccgaagagcgaggtgcgttcctgaaagcagaactccaaaagttggcgcaacagtacccttgcattggaaatgtccgcggtaagggcctgatgatgggctttgagatcgtggatgagcgccaaccgcaagatcgcattggctcttaccctgctgatggcggccttgcggccgaaatccaaaaagcctgcttcaagaacaaattgttgttggaacgtggcggtcgttgcggaaatgttgtccgcattctttgcccactcatcatcacccaggcggaatgtgaagaaatgatcaagcgcttcagcaagtccgtggcagatgctctgaacacggttcgcggt

SEQ ID NO: 39 - DNA, 1356 bp, Pseudomonas putida
atgccacagccactgtacgaattcaccgattctccactgctgcagcgtcagcagcagcaagaatccaacgcacgttcctatccacgtcgtatcccactggcactgcgtcgtgcacgtggcatccacgttgaagatgtggaaggccgtcagttcatcgattgcctggcaggcgcaggcaccctggcactgggccacaatcacccagtggtggtggaagcaatccagcgtgtgctggcagatgaactgccactgcacaccctggatctgaccactccagtgaaggatcgtttcgtgcaggatttgttcggcatcctgccagaggccttgcgtcgtgaagcaaaggtgcagttctgcggtccaaccggcaccgatgcagtggaagcagcactgaagctggtgcgtaccgcaaccggtcgttccaccgtgctggcattccagggtgcataccacggcatgtcccaaggcgcactgaacctgatgggctcccacggtccaaagcagccattgggtgcactgcttggcaacggcgtgcagttcatgccatatccatacgattaccgttgtccattcggccttggcggtgaagcaggcgtgaaggcaaacctgcactacctggaaaacctgctgttggatccagaatccggtgtgccactgccagcagcagtgatcctggaagtggtgcaaggcgaaggcggtgtggtgccagcagatatcgaatggctgaagggcgtgcgtcgtatcaccgaacaggcaggcgtggcactgatcgtggatgaaatccagtccggcttcgcacgtaccggtcgtatgttcgcattcgaacacgcaggcatcgtcccagatgtggtgaccctgtccaaggcaatcggtggctccctgccactggccgtggtggtgtaccgtgattggctggatacctggaagccaggcgctcacgctggcaccttccgtggcaaccagatggcaatggcagcaggctccgcagtgatcaactacctggtggaacaccgtctggcagaacacgccgaagcaatgggccagcgtctgcgtggccacttgcagcgtttgcaacgtgattacccacagctgggcgatatccgtggccgtggcctgatgctgggcgtcgaactggtcgatccacagggccagccagatgcactgggtcaccctccagcaaaccgtgatctggcaccaaaggttcagcgtgaatgcctgaagcgtggcttgatcttggaacttggcggtcgtcacggtgcagtggtgcgtttcctgccaccactgatcatctccgcagagcagatcgatgaagtggcacagcgtttctccgcagccgtggcagctgcagtgggctccgtg

SEQ ID NO: 40 - DNA, 516 bp, Methylomicrobium alcaliphilum
atgcttccagataagaccgcactccctatcattaccttgagccagccaactgcagaagtgggagcccaagtccaccgtttgatctccaaatgcccaccattggacccaaactccatgtactgtaacttgctgcagtcctctcatttctcggaaaccgcggttgcagccaagatcggcgatgaattggtgggcttcgtttccggctatcgtattccgcagcgacccgacaccttgtttgtgtggcaagttgctgtgggcgagaaggctcgtggacaaggtctggccactagaatgcttaaagctatcctcgcgcgtccggtgaaccaggacatcaatcgaattgaaaccactattaccccaaacaacaaggcatcctgggccttgttcgagggtctggcaaagaaacttgacacccagatcggctccgccgtcatgttcgataaaacccgtcactttgctgaccaacatgaaactgagatgctggttaaagtgggtccattcaaggctgttcaggcg

SEQ ID NO: 41 - DNA, 516 bp, Methylomicrobium alcaliphilum
atgctgccagacaagaccgctcttccgattatcactctctctcaacccacagccgaagtgggtgcgcaggtgcaccgcttgatttcaaaatgcccaccactcgaccccaattccatgtactgcaacctccttcaatcgagccacttctccgagactgcagttgccgcgaagattggtgacgaactcgtcggtttcgtttctggttaccgtatcccacagcgtcctgatacccttttcgtgtggcaggtggctgtgggcgaaaaggcacgcggccagggactggccacccgcatgttgaaggcaatcctcgcacgtccggtgaatcaagatattaaccgcattgaaaccaccatcacccccaacaacaaagcatcttgggccctcttcgagggactcgctaagaaactcgacacccaaatcggttcggccgttatgttcgataagacccgccacttcgcggaccagcacgaaaccgaaatgcttgtcaaggtcggaccctttaaggccgtgcaggct

SEQ ID NO: 42 - DNA, 549 bp, Desulfurispirillum indicum
atgatcgcggataagaaacacttcgaacgctttaccatgagacagaccggcgataccgatccatccaccatcactttgaccaccccaaccctggcagatggcgccgaagtctaccgtcttatttctcagtgcccaccattggacgtgaactcccaatactgctatcacatcatttgtgagcatttcagccaaacctctgtgatcgcccgtattgatggcgacgcagccggtttcatctcaggatataccttgccggaacatcccgacatcctgtttatttggcaggtggccgtctctgctaaggcgcgtggtcgacaacttgctacccgcatgctcgagcacatcatttgtcgcaaaagagcaactgccgtccgattccttcataccaccatctccccatctaatattccttcgcagaagctgttctccgctatcgcgcgtacctttaaagcaccgatgcgtgaacgagagttcttggctggcgaagtttttgcggagggtcacgaagcagaggtgttgtacgagattggccctctgcagtatagccaa

SEQ ID NO: 43 - DNA, 549 bp, Desulfurispirillum indicum
atgatcgcggacaagaagcacttcgaacgcttcaccatgcgtcagactggcgatactgatccctctaccattacccttaccacaccaaccctggcagatggcgcggaagtctaccgcctcatctcccaatgtccgccgttggacgtcaactctcaatactgttaccacatcatctgtgaacattttagccaaacctctgtaattgcccgcatcgacggagacgccgctggatttatttccggatacacactcccagaacaccccgatatcctcttcatttggcaggtggccgtgtcggccaaggctcgaggccgccagctggcaactcgaatgctggaacatatcatctgtcgcaaacgtgcaactgcagttcgattcctgcacaccaccatcagcccctcaaacatcccctcgcaaaaactgttcagcgcaattgcacgtacttttaaagcaccaatgcgtgaacgcgaatttttggccggtgaggtcttcgcagagggccacgaagccgaagttctctacgagatcggaccgctccagtattctcag

SEQ ID NO: 44 - DNA, 396 bp, Unknown
  Description of Unknown hydrothermal vent metagenome sequence
atgatcgtccgtacccttaaggattgcaagaactccgaacgagcaatcaacggcggtacctgggaatccgtgcgtatgttgctgaagaacgacaatatgggcttctcctttcacatcaccactattttcgctaacaccgaaaccccaatctggtaccagaatcacttggagtccgtgtattgcatgtctggacgcggcgaagtggagaccgtggctgatggcaagatctacccgattgaacccggtaccttgtatattctggataagaacgacaaacacttgttgagagcatctgaagatatggagatggcctgtgtctttaacccacctttgaatggtaaagaagttcatgacgagaacggtgtgtacgctcttgatggagagaccctcactgac

SEQ ID NO: 45 - DNA, 396 bp, Unknown
  Description of Unknown hydrothermal vent metagenome sequence
atgatcgttcgcacccttaaggactgcaagaactcggaacgcgccattaacggcggaacatgggagagcgtgcgaatgctgctcaagaacgataacatgggcttttccttccatatcaccacaatcttcgcgaacacggagacccctatttggtaccagaatcatttggagtcggtgtactgcatgtcaggccgcggagaggtggagaccgtggctgatggtaaaatttatcctattgagcctggaaccttgtacatcttggataagaatgataaacatctccttcgagcttctgaggatatggagatggcctgtgttttcaatccaccattgaacggtaaagaggttcacgatgaaaatggtgtgtatgcgttggatggcgagaccctgactgat

SEQ ID NO: 46 - DNA, 396 bp, Alkalilimnicola ehrlichii
atgaagattgttaaattgcaagatatcattggcactgagcgcgaagtacatggagaaggctggcgctctcgccgtatcctcctgaaaaaggacaaaatgggtttctccctccacgagactatttggccacccggtcacgagttgcgtatgtggtataagaatcatttggaagcagtatactgcgttgcgggaaacggaagcatcgaggatcttgagacaggtgaggttcatgaactttatgatggcgtgtgctacgcacttgataagcacgatcgacacatcctgcgcggcggtacagaagaaatgcgtctggtgtgtgtattcaacccgccggtgacgggactcgaagtgcacgatgaagatggagcgtactgcctcgttgaagatgacgacgac

SEQ ID NO: 47 - DNA, 396 bp, Alkalilimnicola ehrlichii
atgaagatcgttaaactgcaggacatcattggcaccgaacgtgaagtgcacggcgaaggctggcgatcccgtcgaatcttgctgaagaaagacaagatgggcttctctttgcacgaaaccatctggccacctggtcatgagcttcgtatgtggtacaaaaaccacttggaggcagtttattgcgtggccggtaatggctccatcgaagatttggagaccggcgaagtgcatgagctgtacgacggtgtctgttatgctcttgataagcacgaccgccatattctccgtggcggcaccgaagagatgcgtttggtgtgcgtctttaacccgccagtcactggactggaagttcacgatgaggacggtgcatactgtcttgtcgaagatgacgatgac

SEQ ID NO: 48 - DNA, 510 bp, Methanobacterium formicicum
atgggtccggagaacgatgaaaatcagccagttaagatccgcacccctcagattaaagacggaaatcaaatctaccacttggtgaagaaatctaagcctctcgacatcaactccttgtactcttatttgctgatttgcacccatttcgacgagacctctgctgtcgcggaatggaacaacaagatcattggcttcatctcagcttatattaacccacaccaggagaacaacttgttcatctggcaagtggctgtccttcctaccatgcgtggccagggtctcgcgacccaaatgatcgagcacattcttcatcgaaaggaaacccagtcgatcgagttcattgaaaccactgtgaccccaactaacgatgcatctatggcactgtttaagaacatcgcagccaacttggacaccaaccttgaaaagacctctttcttcactcgtgaattgttcggaaacccgcaccatgaagaggaacttctcttgcaaattggccccctgaacaacaag

SEQ ID NO: 49 - DNA, 510 bp, Methanobacterium formicicum
atgggcccggagaacgatgagaatcagccggtgaagattcgaactccgcagatcaaagacggtaaccagatctaccacctcgtcaaaaagtccaagcctctggatattaactctctgtattcctatctcctgatctgcacccacttcgatgaaacatcggcagtcgcagaatggaataataaaatcatcggcttcatctccgcctatatcaatccccatcaagaaaacaatctcttcatttggcaggtggctgtcctccccacgatgcgtggtcaaggcctggccactcagatgattgagcacattctgcatcgaaaggaaacacagtccattgagttcatcgaaactaccgttaccccgactaacgatgcctccatggcattgtttaaaaacatcgctgctaacttggataccaatctcgagaaaacgtccttcttcacacgtgagctcttcggcaatcctcatcacgaggaggagttgttgcttcagattggcccgctcaataacaag

SEQ ID NO: 50 - DNA, 396 bp, Hahella chejuensis
atgatcgtgcgcaccctcgaacaagcacgacagtcagaccgtcgcgtcaaagcagacaattgggaatctgtgcgcatgctgctgaaggatgacaatatgggcttttcctttcacattactaccctgtacgcaaataaggaaacaccaatccactatcagaaccacctggaatcagtttactgcatttctggcgaaggtgaagtggagacagtggccgatggagaagtgcacgctattaagcctggcaccctctatgtgctcgacaagcatgataagcacctgctccgcgccttctctgagatgaccgtggcatgtgttttcaacccgccattgaacggtaaggaaacacacgacgaaaacggagtgtacccacttgaggctgaaaccatcatggaa

SEQ ID NO: 51 - DNA, 396 bp, Hahella chejuensis
atgatcgttcgcaccttggagcaggcaagacaaagcgatcgtcgagtcaaggccgacaactgggaatccgtgcgtatgttgctgaaagatgacaatatgggcttctcctttcacatcaccactctttacgctaacaaggagaccccgattcactaccagaatcacttggaatccgtgtattgcatctctggcgaaggtgaggtggaaaccgtcgcagatggcgaagtgcatgccattaaacccggcactttgtacgtcctggataagcacgacaaacacttgttgcgtgctttctctgaaatgaccgttgcgtgtgtgtttaacccacctcttaatggcaaggagacccacgacgaaaacggagtctatcctctggaggccgaaactattatggag

SEQ ID NO: 52 - DNA, 513 bp, Methanosaeta sp.
atgcgtgcctccttgcgtcgcactaaagaatttgttttgcgtaagccggaagtttccgacggtgcgaaaatcttcaaccttgtcgaaatctgcaagccacttgacttgaacagcgtatactcttacctccttctctgccatcatttcgcagatacctgcgttgtcgcggaattggatggtgaaatcttcgctttcctcagcggataccgccccccagatcaggaaggtgtatttttcgtttggcaagtcgcagttgatccacagatgcgcggacagggacttggtaagcttctcctgaccgagacactcaagcgacctgccgttcgcggctgcaatttccttgaaaccactatcaccccatccaacaacgcgtcgcagcgtctctttacgtcattggctcgtgatcttaaggctcgctgcacagtgtcatcatgtttttcagatgagcactttggtcaggaaaaccacgaagctgagcacctgtttcgtattggaccgttctcgctctccggc

SEQ ID NO: 53 - DNA, 513 bp, Methanosaeta sp.
atgcgcgcatccttgcgtcgaaccaaggagttcgtgttgagaaaaccagaggtctctgatggcgcgaagatctttaacctggttgaaatttgcaaacctcttgatttgaactccgtgtactcctatttgctgctttgccaccatttcgctgatacctgtgtggtcgcggaactggacggagagatcttcgcatttctttccggctaccgcccacctgatcaggaaggcgtgttcttcgtttggcaagttgccgtggacccacagatgcgaggccaaggtcttggcaagttgttgctgaccgaaactttgaaacgcccggcagtgcgtggctgcaacttcttggagaccactatcaccccatctaacaatgccagccagcgtttgttcacctctttggcacgtgatcttaaggcccgatgcaccgtgtcctcttgtttcagcgacgaacactttggtcaagagaaccacgaagcagagcacttgttccgtattggcccattctccctgtcaggt

SEQ ID NO: 54 - DNA, 390 bp, Unknown
  Description of Unknown Phenylobacterium zucineum sequence
atgatcgttcgtgatctgtcccaggcagaaaagaccgaccgtcgagtggtgtccaaaggatgggagtcaactcgtttgctgcttaaggatgacgcaatgggcttctcctttcacatcaccactatctacgaaggtgcagagctgccgatgcactacaaaaaccacttggaagccgtctattgcatctccggcgaaggttctattgaggatttggctaccggacaggtccatgagattcgtcccggcgttctctacgcgttggataagcacgaccgacatgtgcttcgcggtagaacccaaatggtcatggcatgtgcattcaacccaccagttaccggtcgcgaagtgcacgacgagactggagcatatcctttggaagctgagatggcg

SEQ ID NO: 55 - DNA, 390 bp, Unknown
  Description of Unknown Phenylobacterium zucineum sequence
atgatcgtccgcgatctgtcccaagccgaaaaaaccgatcgtcgtgtggtttccaagggctgggagagcacccgccttctgcttaaagatgacgcgatgggcttctcttttcacattaccacgatttacgaaggagcagagctcccaatgcactataaaaatcacctggaagctgtgtactgtatcagcggtgaaggttcaattgaggatttggcaaccggtcaggtccatgaaattcgcccgggcgtgctgtacgccctggacaaacatgaccgacatgtcctccgcggacgcacccagatggtgatggcctgtgcattcaatccgcctgtgaccggccgtgaggtacatgatgagaccggcgcataccccctcgaagcggaaatggcg

SEQ ID NO: 56 - DNA, 528 bp, Methanothrix soehngenii
atgaaatccgatcagggttcctcagactcagaagacgttggtcgcctcaaattccgtaaaccgcgccttaatgatggtgcgcgtatctctgaactgatccgttcctgcaaacccctggatattaactctacttattgctacctgctgctctgtcgcgatttcgcagatacgtgtgtcgtggctgaagaaggtggtgaaattgtggcgttcctttctggataccgccctccggatcgctgggatgtgttcttcatttggcagatcgcggtagacgttgattcgcgcaaccgtggcatcgcgaaacgtatgctgttcgatgtcttgaatcgtccaagcacccgtgattgccgatacattgagaccactatctctccatcaaatgaggcctccaaacgcttgtttcgcggattggcacaagaactggatgcccactgcgatgtttcagattgcttttccgaaagcgattttggtgacttcgaccacgaggccgaatgcctgtttaacatcggcccatttaagatgaacggt

SEQ ID NO: 57 - DNA, 387 bp, Bacillus halodurans
atgaaggtggtcaaactggaagatgttatcggtaccgaacaggaagtgaagggagagaactggacctctcgtcgattgctgcttaagaaagatggcatgggttactctgtgcacgacaccatcattaaagctggaaccgaaactcacatctggtaccaaaaccacttggaggcggtctattgcattgaaggagagggcgaagtcgagaccgttaaggacggtaaagtttggccgatcaaggcaaatgaaatctacgcccttgatgaacacgacgagcacttgttgcgcgcaaagaccgatatgcgtatggtttgtgtgttcaacccacctattaccggcaaagaaactcacgacgagaatggtgtgtatccattggtcgatgacgag

SEQ ID NO: 58 - DNA, 387 bp, Bacillus halodurans
atgaaagtcgttaagctggaggatgtcatcggaactgagcaggaagtgaagggtgaaaactggaccagccgccgcctgttgttgaagaaggacggtatgggctactccgtgcatgatactatcattaaagcaggcaccgaaacccatatttggtaccagaatcacctggaagccgtgtattgcatcgagggagaaggagaagtcgaaacggtcaaggacggtaaggtgtggccgatcaaagcgaatgaaatctatgcgctcgacgagcatgatgaacacttgttgcgcgccaaaaccgatatgcgaatggtttgtgtgttcaacccccccatcacaggcaaggaaactcatgacgaaaacggtgtgtatcccttggtcgatgacgag

SEQ ID NO: 59 - DNA, 501 bp, Unknown
  Description of Unknown Nitrosopumilus maritimus sequence
atgacctctatcacttttcgagaaccacgagttgacgatgcgaacaagatttgggaactcgtccagaataacaaaccactggatgaaaatagcaaatacctttacgtattgctctgtcaccaattctcgaaaacttgcgttgtggcggagtacgactctaagatcgtgggcttcctgtcgggcttcatctcaccaaagaaccatgatactctcttcgtatggcaggctgcggttgatgattcgtaccgcaacaaaggcgtcgctaaggagttggttttcaaagcattctcccaaactgattacgacgtccgcttcgtggaggcgactgtaacaccatccaacaaggcctcacttaagttcctgcagaatttcgccgcgcaattggacgcagactttgtcaagaccccactgttctcgtccgatgtactgggcgatggacacgaacaagaagatctggttcgcatcggcccaatccaaaaatctcttctggaagtcaccgcc

SEQ ID NO: 60 - DNA, 501 bp, Unknown
  Description of Unknown Nitrosopumilus maritimus sequence
atgacctctatcaccttccgtgaaccacgagtcgatgacgcaaacaagatttgggaattggttcagaacaacaagcctctggatgaaaactccaaatacctttatgttttgctgtgccaccaattctctaagacctgtgtggtcgccgagtacgatagcaaaatcgtcggcttcttgagcggctttatttcaccaaagaaccatgacactttgtttgtctggcaggcagccgttgatgactcataccgcaacaagggtgtggcaaaagaactggtcttcaaagccttttctcaaaccgattatgacgttagattcgtggaggctaccgtgaccccatcgaacaaggcgtccttgaaattcctgcagaattttgctgcgcaactggatgctgacttcgttaagaccccattgttctcctctgatgtgctcggcgacggtcacgaacaggaagatttggtgcgtatcggccccattcaaaaatcccttttggaagtgaccgca

SEQ ID NO: 61 - DNA, 411 bp, Sphingopyxis alaskensis
atgatcgtccgtaacctgggcgacatccgaaagaccgaccgtaatgttcgatccgatggttgggcttctgcgcgcatgttgctgaaagatgacggcatgggcttctcctttcacgtgaccactttgttcgcaggctccgaactgcgaatgcactaccagaaccacttggaggccgtgcttgtcctcaagggaaccggcactatcgaagaccttgctaccggtgaggttcatgcgctccgtccaggcgtgatgtatgctttggatgaccacgatcgccatatcgtgagacctgaaaccgacattctgactgcgtgcgtgttcaacccaccagtgaccggccgcgaagtccacgatgagtccggtgcatacccggccgaccccgaacttgcaagagagccagtcgcagccgat

SEQ ID NO: 62 - DNA, 411 bp, Sphingopyxis alaskensis
atgattgtgcgcaacctgggcgatattcgtaaaaccgatcgaaacgtacgcagcgacggttgggcatccgcacgtatgttgctgaaggatgacggtatgggcttctcctttcacgtgactaccttgttcgctggcagcgaactgcgcatgcactaccaaaaccacctggaagcagtgttggtactcaaaggaaccggcactattgaagaccttgcaaccggcgaagtacacgccttgcgcccgggcgttatgtacgccctggacgaccacgatcgacacatcgtgcgcccggaaaccgatattcttaccgcgtgtgtctttaatccacctgttacaggccgtgaggtgcacgatgaaagcggcgcataccctgccgatcctgagctcgctcgtgaaccagttgcagcggat

SEQ ID NO: 63 - DNA, 831 bp, Unknown
  Description of Unknown Candidatus Entotheonella sp. sequence
atgctcattcgcacgatggatcaattggaggctgagggccgagtcatttccatctcgcatggccgcgccaccgcggtgcgcctgttgacgaagagcgacggtctgggtttttccgtttcagaagcccgcgccacacaagcaggacaaagcgatctctggtacaagcaccactgggaagcaaattacgtgcgcagcggccgtgccaccttggaggaccgcagcacaggcgaacgttggcaactggagccaggtgttctctactgcgtaggaccgcgcgatcgccaccgtattattcgtgatgaaccatccgatatgcgtatcatttctgtcttcaaccctcctatcgaaggtgatgagacacacgacgcggatggcgcatatcctcccaccggagaacctccccgcggacaggatcgtatgttcgtgaagaccctggcagatgttcgccaggcgggcatggagcgcaccgtggctaatggcaaggctgtgtccgcccgctacttgaccgttgacgatcatctgggtttctccctcaactccgttcaccttaaggccggatccgaagcagatctctggtacaagcaccattgggaagccaatctcgtgctcgatggcaccctggatgttattgattatgcaacgggcgatgttcaccgccttggtcccggaggtctgtacctggtgggccctaaggatcgccaccatgtcaaggttctctctgattttcacggcttgtccgtttttaaccctccgctcaccggcaccgaagatcacgatgccgatggctcgtaccccccgactggcccagttcccccaggtcccgcgggtggc

SEQ ID NO: 64 - DNA, 831 bp, Unknown
  Description of Unknown Candidatus Entotheonella sp. sequence
atgttgatcagaactatggatcagctggaagctgagggacgagtcatctccatttctcacggcagagcaaccgccgttcgtttgctgactaagagcgacggtttgggcttctccgtgtccgaagctcgtgctacccaggctggccaatctgatctgtggtacaaacaccattgggaagcaaactatgtcagatccggccgtgccaccttggaggaccgatctactggtgaacgctggcaattggagccgggtgttctgtactgcgtgggaccccgtgatcgacaccgcatcattcgagacgagccatctgatatgcgcatcatctccgtgttcaacccaccaatcgaaggtgacgagacccacgatgcagacggagcctatccgcccactggcgaaccacctcgaggccaggaccgcatgtttgtcaagaccttggctgatgttagacaagcgggcatggagcgtaccgtggctaacggtaaagctgtctccgcgcgttacctgaccgtggatgaccaccttggcttctccttgaactccgtccacttgaaggcaggttccgaagccgatttgtggtataaacaccattgggaggctaaccttgttctcgacggcaccctggacgtgattgattacgcgactggcgatgttcaccgtcttggtcctggcggtttgtatctggtgggcccaaaggaccgtcaccatgtgaaagtcctttcagatttccacggcttgtccgtgttcaacccaccattgaccggaactgaagatcatgacgcagatggttcctacccacctaccggaccagttccgcccggccctgcaggcggc

SEQ ID NO: 65 - DNA, 501 bp, Flexistipes sinusarabici
atggaggataacatcatcatcaagcgcccggtggtgaaagacgctttcggaatttggacgctcattaaaaactcagaacctctggatctgaattcgaagtacgcctacatgctgttctgcacccactttcgcgacacctcagtagtggcaattgacccagatcaggacgataaagtcgtgggtttcgtctcaggctatcgccctccagttcaaccagacgcccttttcgtatggcaggttgtggtagactcctctatgcgaggtcgcggcctcgcacctcgcattattattgatatccttgaacgtgaatacaatctggatatcaactacatccaagcaacggtgggcccgtctaataagccctctgaaggtctttttcgtaaactcgctaagtactatgatgcaaagtgtgaaacctcagtcttcttcagcaaagaggacttcggcgagggagaccatgaagaggaggtgctctttcacattggcccaattaaacgcggt

SEQ ID NO: 66 - DNA, 501 bp, Flexistipes sinusarabici
atggaggataacatcattatcaagcgcccagtggtcaaagacgctttcggcatctggacccttattaagaactctgaacctttggatttgaactccaaatacgcttatatgctgttctgcacccactttcgtgacacctctgtggtggcgatcgatccggaccaggatgacaaggtcgttggtttcgtgtccggctaccgtccaccagtccagcccgatgcactttttgtttggcaagtggtcgttgactcctctatgcgtggccgtggcttggcaccacgtattatcattgacatcttggagagagaatacaacctggacatcaattatattcaagccaccgtgggaccgagcaacaagccctcagagggtcttttccgtaagttggcaaaatactatgatgccaagtgtgaaacctctgtgttcttctctaaagaggatttcggagaaggcgaccacgaagaggaagtcttgtttcatatcggccctattaaacgtggt

SEQ ID NO: 67 - DNA, 561 bp, Leptospirillum ferriphilum
atgaattctgagaacatcagcccggttctcagcgacttccaagccccctcgcgccgtgaaaccgagcgtctcttccgattccgtacgccgaccgacctcgatggacaatccgtccatcgtttgatcgcacgatgccccccacttgatgtgaactctctgtacgccaacctcctgcagtgcctgcacttctctggtacctccattcttgcggaaacgggagatggccagctcgctgccttcatctccggctacgtcatccccgaaaatccagatacacttttcatctggcaggttgcagtcgatccagtacaccgcggtaagggcgtcgcactgagcatgcttgaggcgcttctcgatcgcactcttagcatgggagttcgttaccttgagaccacaatctcgccggaaaacggaccttctcgcaaactgttccagaagctcttctcccgccgtaaggcgcctttctcacgtcgcccactcttcggccgcgagactcacttcggtggcggtcacgacgacgaggtcctttaccgcggtggcccattttcggctgtgggccct

SEQ ID NO: 68 - DNA, 387 bp, Unknown
  Description of Unknown Nitrincola nitratireducens sequence
atgattgttcgccacctgaaggattgcgaagcaagcgaacgccgcgtggtgtccgaaaactgggagtctacccgcatgctgctgaaagacgatcagatgggcttctcctttcacatcacgacaatctacgccaacaccgaaacccatatctggtatcaaaaccaccttgaatccgtttactgcatgtccggcaccggtgaaatcgagacgcttgctgacggaaaggtctatcctatcgaaccaggcatgctgtacatccttgaccaacacgatgagcacttgctccgagccactactgaaatgaagatggcttgcgtcttcaatcctccccttagcggcaaggaagtgcacgatgagaatggcgtttatccagtggatgactccaag

SEQ ID NO: 69 - DNA, 387 bp, Unknown
  Description of Unknown Nitrincola nitratireducens sequence
atgatcgtgcgccacctgaaggattgcgaagcatctgagcgtcgagtggtctccgaaaactgggagtctaccagaatgttgctgaaagatgaccagatgggcttctcctttcatatcaccactatctacgctaacaccgaaactcacatctggtaccaaaatcacttggaatccgtgtattgcatgtcaggcaccggtgaaattgagaccctggcggatggcaaggtctacccgatcgaacccggcatgttgtatattctggatcagcacgacgaacacttgttgcgagcaaccactgagatgaagatggcctgtgtcttcaacccacctttgtcgggtaaagaagttcacgacgagaatggagtttatcctgtggatgactccaaa

SEQ ID NO: 70 - DNA, 480 bp, Nitrospina sp.
atgcaactgcgcaaacccagcctggaagacggccctcgtatgtttgacctcgcacgctccgccggtaacctcgacgtcaactcttgttaccactacttgctgttgtctcagaagttcgcagagacctgcgcagtggcagtgcagggcgagaagctgatgggcttcgcaaccggcttccgcgaccccaacaagccagagatcctgttcatctggcaagtggcagtctctgatgactttcgtaacggcggagttgccaaggccatgctcaaccacatcctccagcgtaacttttccccgcctattcaattccttgaagtaaccattgcgccgagcaacaaagcctcccaggctctgttccagtccctggcgaacgaactcaactgcagcatcgaaaacgaatctttgatctccgccgacctcttcccggaaaacggtcacgaacctgaatcactgttccgcattggtcctcttaactgccat

SEQ ID NO: 71 - DNA, 396 bp, Marinobacterium stanieri
atgatcgttcgcaccttggaagagtgcgagaactccgaacgtcgaattgtgtccaagacctgggagtctactagaatgttgctgaaagatgacaatatgggctactctttccacatcaccactatctacgcaggtaccgagactcacatctggtaccagaaccacttggaatccgtgtattgcatctcaggcgaaggcgagattgaaaccctggctgatggcaagatctacccgattaaacccggcaccctttataacttgaatgagcacgatgaacacttgttgcgagctgacaaggaattgaaactggcgtgtgtctttaacccacctttgaatggcaaggaagtgcacgatgaaaacggagtctaccctctggaggcagacaccgttgaagcc

SEQ ID NO: 72 - DNA, 396 bp, Marinobacterium stanieri
atgatcgtccgaaccctcgaggagtgcgaaaactccgagcgccgcatcgtgtccaaaacctgggaaagcacacgcatgttgctgaaagatgataacatgggctactcctttcatatcacgactatctatgcaggcactgaaacccacatctggtaccagaaccatctcgaatctgtgtattgtatttctggtgaaggtgagatcgaaacccttgcagatggcaaaatctaccctatcaagcctggtaccttgtacaacctgaatgaacacgatgaacaccttctgcgcgctgataaggagctgaaactggcctgtgtcttcaaccctccgcttaacggcaaggaggtccacgatgaaaatggcgtgtaccctcttgaagcagacaccgttgaagct

SEQ ID NO: 73 - DNA, 522 bp, Spirochaeta africana
atgaacttccgccaccccactgctgaagatggtgccgccatctggcgcattgtccgcgactctggtgttctggacctgaatagctcatattgctaccttatgatgggacactacttcggtgatacctgcatcgttgcagaggatccacataccgacccagctgcgaatggcgatcgtctcgcaggtttcatcgtgggctttcgaccgccgcaagacccagccaccttgttcgtatggcaagtggccgtggacccgggccatcgcggcaagggcgttggcagccgcatgctgcaggcattgcttgcagcacaaccagcagaggagatccattacatcgaagctacgatctccccgtccaacactgcttcccaagctctgtttcgcaaggctgctgccaccctcaagactgactgttccgtttcagatctgtttcttgctgacatgttccccggttccggcgaacacgaaccggaacaccgctatcgaattggaccgttcacgctgtctaactccgccaatcag

SEQ ID NO: 74 - DNA, 393 bp, Actinomadura sp.
atgatcgtgcgttccttgaaggaaatcattggaaccgagagagatgtccaggctcctacttggcactcccgtcgattcgtcttggcgaaggacggcgttggtttttctatgcatgaaaccgttctgtacgcaggtaccgagactaaaatgtggtacgcaaaccacatcgaagccgtgtattgcattgaaggacaaggcgagcttatcaaccacgaaaatggcgagacccatcgtattgaaccaggcgtgatgtacttgctgaacgagcacgatcaccataccctgcgagcacataccgacgttaaaactgtgtgtgtcttcaacccacctgttaccggccgtgaagtgcacgatgagaatggtgtgtatccccttctcactgaagaggacgcc

SEQ ID NO: 75 - DNA, 393 bp, Actinomadura sp.
atgatcgttcgatcgctcaaagagatcatcggtacagaacgtgatgtccaagccccaacttggcactcacgccgcttcgtcttggctaaggatggtgttggtttctcaatgcacgaaacagtcctgtacgcgggtacggaaaccaagatgtggtacgcgaatcacattgaagccgtttactgcattgaaggtcagggtgaactgattaaccacgaaaacggagagacccaccgcattgaaccgggtgtgatgtatctccttaatgaacatgatcatcacaccctccgtgctcacaccgacgtaaagaccgtgtgcgtgttcaacccgcctgtgaccggccgtgaagtccatgacgaaaacggagtctaccccttgcttaccgaggaggatgcg

SEQ ID NO: 76 - DNA, 501 bp, Dehalogenimonas lykanthroporepellens
atgaaaatcaattaccgccagccggacattaaagatggcttggatatttaccgcctcgtgcagtcctgcccaccgctcgaccttaactccgcttactgctacctccttctggcgacccatttcgccgatacctgcgtcatcgcagaggatactaccgcgggcaaaatcgcgggttttgtgtcggcgtacttccgcccagacgcgcctcgcaccctgttcgtctggcagatcgcggtgcatcccgactaccgaggccaaggtatcgcacgcggtctgactactgcgcttgttgaagccgcttaccgccagacaactcccccggagtacctcgaacttagcgtaaacccatccaaccaggcttctcgaggtttgttcacgcgcttggctgagcagctcggtgccgagctgaccgaatcggtcttgttctcggaagaattgcttggcggtggccacgaggcggagatcatgctccgcattggccctctgaaaggtgatccacaa

SEQ ID NO: 77 - DNA, 501 bp, Dehalogenimonas lykanthroporepellens
atgaagatcaactaccgccagccagacatcaaagacggtctcgacatctacagactggtgcaatcctgcccacctctggatttgaactccgcatactgttatttgctgcttgctacccacttcgcggacacttgcgttatcgccgaagataccactgcaggcaagattgccggcttcgtgtcagcatactttcgtccagacgcccctcgaaccctgtttgtgtggcagatcgccgtccatccagattaccgtggccaaggtattgctcgaggcctgaccactgcgcttgtcgaagcagcctaccgccagaccaccccaccagaatacttggagttgtccgttaacccttctaatcaggcatcccgtggcttgttcaccagattggctgagcaactgggcgcggaacttaccgaatccgtgttgttctccgaagaattgttgggcggcggccacgaagctgagatcatgcttcgaattggtccgttgaaaggcgatccacag

SEQ ID NO: 78 - DNA, 393 bp, Leptospirillum ferriphilum
atgatcgtccgcaccctggaagaggctgagagatccgaacgtcgagtggtcaccccaacttgggagtctacccgtttgctgcttaagaacgatcgaatgggcttctcctttcacatcaccactatctacgcaaacaccgagactcacatctgctaccagaatcacttggaagccgtgtattgtatttctggcgaaggtgaggtcgaaacccttgctgatggagttgtgcataaaatccgccctggcactatctacgttctggaccgtcacgatgaccacttgttgcgtggtatcaccgagatgaagctggcgtgcgtgttcaacccaccattgagcggtaaagaagtccacgacgaagaaggcgtgtatccgttggaggcagaaccaatctcc

SEQ ID NO: 79 - DNA, 393 bp, Leptospirillum ferriphilum
atgatcgtgcgtacccttgaggaggcggagcgctccgaacgtcgcgtggtcactccgacctgggagtccacccgcctcctcctcaaaaacgaccgcatgggcttctcttttcatatcacgactatctatgcgaacacagagacacacatttgttaccagaaccatctcgaagctgtgtattgcattagcggtgagggagaagtcgagaccctggctgatggcgtggtccacaagatccgccctggtaccatttacgtgcttgatcgccacgatgatcaccttctccgcggcatcacggaaatgaaacttgcatgcgtttttaatccacccctctccggcaaggaagtccatgatgaagagggtgtctatcctctcgaggccgaacctattagc

SEQ ID NO: 80 - DNA, 558 bp, Bordetella bronchiseptica
atgcgtaaggatgaaacctctaacacctctccagacatctccgcagcacagcctgcatcagcactgcgctaccaacttcgtccaccacgtcgtaacgatggtgctgcgattcaccagttggtgtccgagtgcccaccattggatttgaactccctttacgcatatttgctgctttgcgaacaccatgcacacacctgtgtggtcgccgagtctccaggcggtcgtatcgatggcttcgtgagcgcctacttgttgccgactcgtcccgacgtcttgtttgtttggcaagttgctgttcactcccgtgctcgaggtcatagactgggaagagcaatgctcggtcacattttggaacgtcaggagtgtcgtcacgtgcgacacttggaaaccactgtcggccctgataatcaggcatcccgtcgtaccttcgctggtttggcgggagaacgtggcgcgcacgtctctgagcagccgttctttgatcgacaagcattcggcggcgcagaccatgatgacgagatgctgcttcgtatcggcccctttacccacccaccacac

SEQ ID NO: 81 - DNA, 558 bp, Bordetella bronchiseptica
atgcgtaaagatgagacctcaaatacctcacccgacatctccgccgcgcaaccggcttccgctcttcgctatcagctccgcccacctcgccgtaatgacggcgccgctattcaccaactggtttcggaatgtccaccattggacctcaattctctctacgcatatctcctcctctgcgagcaccatgcccacacctgtgtggtagcggagtcccctggtggtcgaatcgatggttttgtatcagcgtatcttctgcctactcgaccggacgtcctttttgtgtggcaggtggccgttcactcgcgagcgcgtggacaccgtctgggtcgcgccatgttgggccacatcctggaacgccaagaatgccgtcacgttcgccacttggaaacgacggttggtccagacaaccaggcgtctcgccgaactttcgcgggccttgcgggcgaacgaggcgcacacgtgagcgaacagccattcttcgaccgtcaggccttcggcggagcagaccacgatgatgaaatgctgctgcgcatcggcccatttacacaccctccacac

SEQ ID NO: 82 - DNA, 408 bp, Unknown
  Description of Unknown Betaproteobacteria bacterium SG8_41 sequence
atgatcctccgttccttggatgaagtgaagggcacctctcgagacgttgagaccccaaactgggtgtcccgtcgattgctgcttcgctccgatggcatgggtttctctctgcacgaaaccactgtctaccctggtaccgagacttacatctggtatgcaaatcacttggaagccgtctattgcgttgaaggagagggcgaagttgagaccattccagacaagaaaatctacccgattcgacccggcgtggtctatgctctggatcagaacgacaagcactaccttcgcgcgagaaccaaaatgcgcctcgtttgtgtgtttaacccacctcttaccggtcgtgaagtgcatgatgagaatggagtgtacccactggtgaccgatgaggcagctgctgctcgtgcttct

SEQ ID NO: 83 - DNA, 408 bp, Unknown
  Description of Unknown Betaproteobacteria bacterium SG8_41 sequence
atgattttgcgttccctggatgaggtcaaaggtacctcccgcgacgtcgagactccaaactgggttagccgccgccttctgcttcgctctgacggtatgggcttttcacttcacgaaactaccgtatacccaggcacggaaacctacatctggtacgcgaaccacttggaggccgtatattgtgtggaaggcgagggcgaggtggaaacgattccagataaaaagatttacccaattcgccctggagtcgtgtacgcgctcgatcagaacgataaacactatttgcgagcgcgcactaagatgcgactggtttgcgtatttaatccgcccctcaccggtcgtgaagtgcacgatgagaacggagtatatccgctggtaacggatgaagcagcagccgctcgtgccagc

SEQ ID NO: 84 - DNA, 825 bp, Unknown
  Description of Unknown hydrothermal vent metagenome sequence
atgttcattcgctctctccgtgatgttaaagatactgaccgctataaagtgctgccgaaatcggccacacacacagctcgatacttgactgctgcagatggtatgggcttctcactccatgttaaccgcggaaaggcaattcccccgacccagctctggtataagaaccactgggaagccaattacctcattgcgggtgtgattaaggttaccgatatgacgtcaggacaggagtggttgcttgaatctggtgatctgtaccaagtgggccctaacgatcgtcatttgtatgaagtcctggaagatgagcaccacctttccattttctgtccttcactgtggggcgatgaaacccacgatgaagatggagcatatagcgaatctggccccgttccagtgaccgaacaacgaatgttcgttcgccacatcgagcacatgcgcgaagcgggtcgcgagatgctggttgccaatggccaggcccgtgtcgtccgcgctctgaccaaaaccgatggcttgggattctccttctccgatgttaacttcgctgctggcgcaacgacgaagctctggtacaagcaccattgggaaaccaactatatcatttctggaaccggccaggttaaagatcttaccaccggcgaatcgtggaagctggagccagacatgacatacaatgttggtccaaaagatcagcaccaactgtccgctgagaccaacctgcacctggctagcgtcttttgtccgccattggagggaaccgagcagcacgattctgacggcgccctcgaaccctccggcccagtcccgccaggtcccccaggttat

SEQ ID NO: 85 - DNA, 498 bp, Unknown
  Description of Unknown Deltaproteobacteria bacterium ADurb.BinA014 sequence
atgaagaacgattcgcaccacatcttgttccgccttcccgaaaagaaggatgcccaggcggttcgccgcctgatcgaggattctcctccgttggaccttaattctctgtattgttatctgcttctttgcactcattttgcccctacctccgcaattgctgagtaccaccaggaagtttgtggctttgtctccgcttatattcgaccggatcagaaagacaccctcttcgtatggcaagttgcggtgcagcgcgactttcgcggaaagtcgatcgctaaaggtatgctgctctcctgcctgcagcgtgcccacttgtcctccctgcgctatctcgaaacaaccgttaatccgagcaacacctcctccaatgcactgtttagctccctcgcagcatttctgaatgctggcctcaccaagagcgtcctgttttccgctgacgactttggcaagatgagccatgaagaagagattttgtaccgcattggcccattcaacatc

SEQ ID NO: 86 - DNA, 567 bp, Bacillus halodurans
atgcaggttcaatgcaatgaaaaggcttttaagggcggctttattattaactctcaaatcgcaacggcaccgcctaaaactctggataccaccatcaccatcggtaagccaaccgttgaagatggagccgccatgtgggagcttgttaacaaaagcacgctggacacgaattctccatacaaatatatcatgatgtgcgaatacttcgcagagacctgtgtcgtggctaaagaaaacgaacgacttgtaggtttcgtcaccgcattcattcccccagaacaccaggacgtaatcttcgtttggcaaatcggtgttgacagctcacagcgcggcaagggcctggcaagcaagcttctccaagaactcatcagccgagatatctgttccaacgtgaactacgtggaagcgaccgtcaccccaagcaacaaagcttcgcaggctttgtttcagaagttggctcgcgaatacaacacccagtgcgaagtttcagagtgcttctcagaagatttgtttcccggagatgaccacgaggcagaactcaccttccgtatcggtccgctgcatcca

SEQ ID NO: 87 - DNA, 567 bp, Bacillus halodurans
atgcaggtgcaatgcaacgaaaaggcattcaagggcggcttcatcatcaactcccagatcgctaccgcgccacctaagactcttgataccactatcaccattggtaaaccgaccgttgaagacggagcagccatgtgggagttggtgaacaagtcaaccctggataccaactccccctacaaatatatcatgatgtgcgaatacttcgctgagacctgtgtggtcgcgaaggaaaacgagcgtttggtgggcttcgtgaccgcttttattccgcccgagcaccaggatgtcatctttgtttggcaaattggtgtcgactcctctcagcgtggcaagggcttggcgagcaaattgctgcaagaactgatctctcgagacatctgctccaacgtgaattacgtcgaggcaaccgtcaccccaagcaacaaggcatcccaggcccttttccaaaagttggcccgcgaatataacacccagtgcgaagtgtccgagtgtttctccgaggacttgtttcctggcgatgaccacgaagccgagcttaccttccgtatcggtccactccatcct

SEQ ID NO: 88 - DNA, 384 bp, Bacillus sp.
atgatcgtgcgaaccctggatgagatcattggtactgaaaacgaggtcaaggcagacacctgggccagccgtcgattgctgcttaaatccgataatatgggtttctcttttcacgacaccatcatctacgcaggaaccgagactcacatctggtacaagaaccacttggaagctgtgtattgcgtcggcggtgaaggagagattgaaaccgtctccgatggcaaggtttacccattgaaagacggtactatgtatgctttggatcagaacgacgaacactacctgcgcgcgaccaaggatatgagaatgatttgtgtgttcaacccaccattgaccggacgtgagactcatgataaagacggcgtttatccactggtggaagac

SEQ ID NO: 89 - DNA, 384 bp, Bacillus sp.
atgatcgttcgcactctggatgaaattattggtacagagaacgaagttaaagcggacacctgggcgtctcgccgcctcctgctgaagtctgacaacatgggcttttcgtttcatgacactattatttacgctggtaccgagacgcacatctggtacaagaaccacctggaggccgtttattgcgtgggtggagagggagaaatcgaaaccgtgtcggacggcaaggtgtaccctttgaaggatggcaccatgtatgctttggatcaaaacgacgaacactaccttcgcgcaacgaaggatatgcgtatgatctgcgtgttcaatccaccactgaccggacgtgaaacacacgacaaggatggcgtttacccactcgttgaagat

SEQ ID NO: 90 - DNA, 528 bp, Streptomyces anulatus
atgaccgcagcaccagctgatttcgctcgtgcacgatcggaatttctgtccatcgatgcccctcgtgtggaggatggagctgcgatctggagaattgctagagattcccaggttttggacctgaactcctcttactcttatttgctgtggtgccgtgacttcgcagcaacctctgctgtggctcgtggcgaaaatggcgagccaattgcatttgtcaccggttacgttcgtccagatcgacctcaaaccttggtggtgtggcaggttgccgtggaccaagctcaccgtggcaagggtctcgctgcggcacttctcgatgctctgaccgcacgtgtggctgctgaccaggtgctgtcctccgtggaaaccactatcaccccggataacactgcgtctgaccgcttgttcacctcttacgcacaaagacacgatgttgcattggaaaaagaggtgctgttcgacggtgaactttttccggaagagacccacttgccagaagtgctctatcgtattggacctttcgcaact

SEQ ID NO: 91 - DNA, 528 bp, Streptomyces anulatus
atgactgcagcacctgctgactttgcccgcgcacgctcggagtttcttagcattgacgcccctcgagtggaagatggcgccgctatttggcgtattgcccgtgattcacaagtactggatcttaactcctcctacagctacctgttgtggtgtcgagactttgctgccaccagcgcagtagcacgcggagagaacggtgaacccattgcgttcgtgaccggatatgtgcgccctgatcgacctcagaccttggtggtatggcaggtggcagtggatcaagcccaccgaggaaaaggtcttgctgcagctctccttgatgcactgacagctcgtgtggcagccgaccaggtcttgtcctccgtggaaactactattacgccagacaataccgctagcgatcgactttttacctcctacgctcaacgtcatgacgttgccttggaaaaggaagtcctcttcgatggagagcttttccctgaagagacccatctccccgaagtgctctatcgcattggcccgttcgccact

SEQ ID NO: 92 - DNA, 384 bp, Unknown
  Description of Unknown Tepidicaulis marinus sequence
atgattgtgcgtacgcttgaagaagccgaaaaaagcgatcgcaaggttcagtcaaaaacatgggattccgtccgtctgctgctgaaggacgataacatgggtttctctttccacatcaccaccctctatgcgggtactgagactcgtatgcactacaaaaatcacctggaatctgtgtactgcatggaaggtgaagcagaaattgaaaccctcgcagatggtaaaaagtatgaaatcaaaccaggcaccatctatatcctcgaccagcacgacgagcacattttgcgcgtgcacaaggaagtgaagatggcctgtgtattcaacccaccagtgaccggtcaagaggtccatgacgaatccggcgcctaccctttggttgtagat

SEQ ID NO: 93 - DNA, 384 bp, Unknown
  Description of Unknown Tepidicaulis marinus sequence
atgatcgtgcgtaccttggaagaggcagaaaagtccgatcgaaaagtccagtccaagacctgggactctgttcgcttgctgcttaaagatgacaacatgggcttctcctttcacattaccactctgtacgctggaaccgaaactagaatgcactacaagaatcacttggaatccgtgtattgcatggaaggcgaggcagaaatcgagaccctggccgatggcaagaaatacgagattaagccgggtactatctatattctcgatcagcacgacgaacatatcttgcgagttcacaaggaagtgaaaatggcttgtgtcttcaacccacctgttaccggacaagaagtgcatgatgagtctggcgcgtatccactggtggtcgac

SEQ ID NO: 94 - DNA, 537 bp, Oceanobacillus iheyensis
atggcagagaagctcaaggtggataccgatgaatactactttcgtcgacctacgaaggaggatggcggcccagtttgggaactggttcgcgaaattggtaatctggatttgaactcctcctattcctacgtattgtggtgcgaagtattcgccgaatcctccatcgtggtcgagcacaaagagacgcagcagattgtgggcttcatctctggctttatgcaccctgaaaaggaagataccttgttcatttggcaagtggcagtcagctccacccagcgtggcaagggtctggcaaccaaaatgctgttgcagctccttgagtggaacgagtcagtgaacttcattgaggcaaccgtcgccccgtccaataagccttctaactatctctttttgggtctggctcgcaagatccataccaactggaagatctccgattatttcaaaaccgaccacttcccagctaaggatgaggagcacgaagaggaactgttgttccgcatcggccctatgcgcaagaataagaacaaacgcatgatc

SEQ ID NO: 95 - DNA, 537 bp, Oceanobacillus iheyensis
atggctgagaagctgaaagtcgataccgacgaatactatttccgtcgaccgactaaggaagatggcggtcccgtctgggagctggttcgtgaaatcggcaaccttgatttgaactcctcttactcctacgtgctttggtgcgaggtcttcgccgaaagctcaattgtggtcgagcacaaggaaacccagcaaatcgtgggtttcatttctggcttcatgcatccagagaaagaagataccttgttcatctggcaggttgcagtgtcctccacccaacgtggcaagggcttggcgactaagatgttgctgcagcttctcgagtggaacgaatccgttaatttcatcgaagcaaccgtggccccatctaacaagcctagcaactacttgtttctgggccttgcacgaaaaatccacaccaactggaagatctccgattatttcaaaactgaccactttccagccaaggacgaagagcatgaagaggaattgctgttccgcatcggtcctatgcgtaagaacaagaacaagcgtatgatt

SEQ ID NO: 96 - DNA, 393 bp, Leptospirillum ferriphilum
atgattgtgcgtacgctggaagaggcagagcgaagcgaacgccgtgtggtgacccctacctgggaatctacccgcttgctcctgaagaatgatcgcatgggcttctctttccacatcaccaccatttacgcaaacacggaaactcacatctgttatcaaaatcatctggaagccgtttactgtatctctggcgagggcgaagtggaaaccctggcagacggcgtggtgcacaaaattcgacctggaaccttgtacgtcctggatcgccacgatgaccacctgctccgtggaattaccgaaatgaagcttgcgtgcgtgttcaatccaccactttccggtaaagaggtccacgacgagaaaggcgtttaccctttggaagcagaacctatttcc

SEQ ID NO: 97 - DNA, 393 bp, Leptospirillum ferriphilum
atgatcgtccgcaccctggaagaggctgagagatccgaacgtcgagtggtcaccccaacttgggagtctacccgtttgctgcttaagaacgatcgaatgggcttctcctttcacatcaccactatctacgcaaacaccgagactcacatctgctaccagaatcacttggaagccgtgtattgtatttctggcgaaggtgaggtcgaaacccttgctgatggagttgtgcataagatccgccctggcactctgtacgttcttgaccgtcacgatgaccacttgttgcgtggtattaccgaaatgaagttggcgtgcgtgttcaacccaccattgagcggcaaggaagtgcacgacgaaaagggcgtgtatccgttggaggcagaaccaatctcc

SEQ ID NO: 98 - DNA, 537 bp, Paenibacillus dendritiformis
atgaacgtgaacgtgactccgtccctgccgtgtaccattcgtaacgcacgtaaaggtgatggcaccgaactttggaagctcgtgaaagaatctggaaccctggatgtcaactcggcctattgctacattatgctctgcgattacttcgcagacacctgtatcgtggcagagcacaatcgtaatatcatcggtttcgtgtccgcgttgcgccgccctgatatgcctgaaacgttgttcgtttggcagatcgctgtgcgcgatcgttaccgcggacgcggcattgctgaggccctgttgcgtaccctctacgatactgcatgtcgtgaccaggtacgctttattgagaccaccatcaccccttccaatacggcttcgcagcgcctcttcgcaaaaatggcgaagacatggaagtcagctcttgtcaccaaagtaggattcgcgtcgcagctcttccccgatggcgcccacgaaaccgagaaattgatccgtattggtcctttgcgtcataacacgcacgatcgcatggagaccgtgacc

SEQ ID NO: 99 - DNA, 390 bp, Phenylobacterium sp.
atgatcgtacgtgatttgaacgaagccaatcaaacccgccgccgcgtcgtgagcaaaggctgggagtccacgcgtctcctcctcaaggacgatcagatgggattctcgtttcacattaccactatctatgcaggtgccgaactgcctatgcactaccagaatcacctggagtccgtctactgtatctctggcgagggctctattgaggacaccgctaccggagaagttcacgagattaagccaggtgttatgtacgcgctcgataagcacgacaagcacattcttcgtggcaaaacggagatgaagatggcgtgtgtgttcaacccgccactgaatggactggaggttcatgacgatacgggtgcgtacccattggaagcagaaactgtc

SEQ ID NO: 100 - DNA, 390 bp, Phenylobacterium sp.
atgatcgtgagagatctgaacgaagctaatcagacccgtcgacgcgtggtgtccaagggatgggagtcaacccgattgctgcttaaagatgaccaaatgggcttctcctttcacatcaccactatctacgcaggtgccgaactgccgatgcactaccagaaccacttggagtctgtctattgcatctccggcgaaggttctattgaggacaccgcaactggcgaagtgcatgagatcaagcccggcgtgatgtacgccctcgataagcacgacaaacatattcttcgtggcaagaccgaaatgaaaatggcctgtgtgttcaacccaccattgaatggtttggaagtgcacgatgacaccggagcttatcctttggaagcggagactgtg

SEQ ID NO: 101 - DNA, 97 bp, Unknown
  Description of Unknown Corynebacterium glutamicum sequence
tgccgtttctcgcgttgtgtgtggtactacgtggggacctaagcgtgtattatggaaacgtctgtatcggataagtagcgaggagtgttcgttaaaa